(19) United States
(12) Patent Application Publication
(10) Pub. No.: US 2024/0287057 A1
(43) Pub. Date: Aug. 29, 2024

(54) PROCESS FOR SYNTHESIZING 2-BROMOLYSERGIC ACID DIETHYLAMIDE VIA CONTROLLED HYDROLYSIS OF BROMOCRIPTINE

(71) Applicant: Ceruvia Lifesciences LLC, Greenwich, CT (US)

(72) Inventors: Annalisa Mortoni, Pavia (IT); Eugenio Castelli, Arlate (IT); John M. Wetzel, Albany, OH (US)

(73) Assignee: Ceruvia Lifesciences LLC, Greenwich, CT (US)

(21) Appl. No.: 18/400,508

(22) Filed: Dec. 29, 2023

Related U.S. Application Data
(60) Provisional application No. 63/436,515, filed on Dec. 31, 2022.

(51) Int. Cl. C07D 457/06; A61K 31/48

(52) U.S. Cl. CPC C07D 457/06; A61K 31/48

(57) ABSTRACT

Disclosed herein, inter alia, are a process for the preparation of 2-bromolysergic acid diethylamide (2-Br-LSD), or a pharmaceutically acceptable salt thereof, via the controlled hydrolysis of bromocriptine to form 2-bromolysergic acid, followed by amidation to form 2-Br-LSD, the purified 2-Br-LSD, per se, and pharmaceutical compositions containing the purified 2-Br-LSD, per se, and uses thereof.

### RELATED APPLICATIONS

This application claims the benefit of priority under 35 U.S.C. § 119(e) to U.S. Provisional Application No. 63/436,515, filed Dec. 31, 2022, which is incorporated herein by reference in its entirety.

### BACKGROUND OF THE DISCLOSURE

2-Bromolysergic acid diethylamide (2-Br-LSD) is under investigation for a variety of pharmacological uses, including preventing and treating disorders associated with cephalic pain. The most efficient known synthetic routes for making 2-Br-LSD involve the reaction of lysergic acid diethylamide (LSD) with a brominating agent such as N-bromosuccinimide (NBS). However, variation of the brominating reagents, solvents, catalysts, and temperatures, provides, at best, a mixture of LSD, 2-Br-LSD and various di- and tri-brominated species. LSD is a highly potent hallucinogen. Batches of 2-Br-LSD intended for pharmaceutical use must be substantially free of LSD, for example, with residual LSD levels not more than 0.05%. Unreacted LSD and these di- and tri-brominated species have proven difficult to separate from 2-Br-LSD by readily scalable, economical methods such as extraction, trituration, or crystallization. These impurities can be removed by chromatography, but this is expensive on large scale. Chromatography on silica gel can remove residual LSD. However, removal of the di- and tri-bromo compounds requires chromatography on media other than silica gel, which is even more expensive. The di- and tri-bromo impurities have been identified as having one bromine at the desired 2-position and the other bromine(s) on the phenyl ring of the indole moiety at positions 12, 13 and/or 14 (ergoline numbering).

Furthermore, LSD is listed in Schedule I of the United Nations Convention on Psychotropic Substances of 1971 and is a Schedule I substance under the United States Controlled Substances Act (CSA). LSD's synthetic precursor, lysergic acid, is a Schedule III substance according to the CSA. As a result of the regulated status of these precursors, they are difficult and expensive to source commercially.

### SUMMARY OF THE DISCLOSURE

A first aspect of the present disclosure is directed to a method of synthesizing pharmaceutical grade 2-bromolysergic acid diethylamide (2-Br-LSD), or a pharmaceutically acceptable salt thereof, comprising: (a) filtering a mixture comprising 2-Br-LSD and a solvent; (b) allowing the 2-Br-LSD to precipitate from the filtered mixture; (c) collecting the 2-Br-LSD by filtration; (d) analyzing the collected 2-Br-LSD of (c) for the presence of iso-2-Br-LSD; and (e1) if a batch of the collected 2-Br-LSD of (c) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or (e2) if a batch of the collected 2-Br-LSD of (c) fails to meet the pre-set specification for iso-2-Br-LSD, then purifying the collected 2-Br-LSD and repeating (d) and (e), or discarding the collected 2-Br-LSD.

Another aspect of the present disclosure provides a batch of pharmaceutical grade 2-Br-LSD, or a pharmaceutically acceptable salt thereof.

A further aspect of the disclosure provides a pharmaceutical composition, comprising pharmaceutical grade 2-Br-LSD, or a pharmaceutically acceptable salt thereof, and a pharmaceutically acceptable carrier.

A further aspect of the present disclosure provides a method of synthesizing 2-bromolysergic acid according to Good Manufacturing Practice (GMP) as an Active Pharmaceutical Ingredient (API) or as an intermediate for API manufacturing, comprising hydrolyzing bromocriptine in an environment in which radical survival is inhibited. The compound 2-bromolysergic acid is disclosed herein as a starting material for the synthesis of 2-Br-LSD.

An even further aspect of the disclosure provides a batch of 2-bromolysergic acid, or a pharmaceutically acceptable salt thereof.

The disclosed methods produce 2-Br-LSD in relatively high yield, at a high purity substantially free of impurities, particularly LSD, iso-2-Br-LSD, dimeric species and di- and tri-bromo species (having one bromine at the desired 2-position and the other bromine(s) on the phenyl ring of the indole moiety at positions 12, 13 and/or 14 (ergoline numbering)).

### BRIEF DESCRIPTION OF THE DRAWINGS

FIG. 1 is a Fourier transform infrared spectrum of 2-bromolysergic acid diethylamide hydrobromide (Form 1) prepared according to Example 5 (lower trace) overlayed with that of the reference standard (upper trace).

FIG. 2 is an X-ray powder (XRP) diffractogram of 2-bromolysergic acid diethylamide hydrobromide (Form 1) prepared according to Example 5.

FIG. 3 is a ¹H NMR spectrum (300 MHz) of 2-Br-LSD hydrobromide.

FIG. 4 is a ¹H NMR spectrum (300 MHz) of 2-Br-LSD hydrobromide, after D20 exchange.

FIG. 5 is a ¹³C NMR spectrum of 2-Br-LSD hydrobromide.

FIG. 6 is a ¹³C NMR spectrum of 2-Br-LSD hydrobromide, expanded from about 10 ppm to about 70 ppm.

FIG. 7 is a mass spectrum of 2-Br-LSD hydrobromide.

FIG. 8 is an XRP diffractogram of 2-Br-LSD hydrobromide (Form 1).

FIG. 9 is a DSC curve of 2-Br-LSD hydrobromide (Form 1).

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 9%, 8%, 7%, 6%, 5%, 4%, 3%, 2%, 1%, or even less than 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the term “batch” refers to a specific quantity of a drug or other material (e.g., 2-bromolysergic acid diethylamide, 2-bromolysergic acid) that is intended to have uniform character and quality, within specified limits, and is produced according to a single manufacturing order during the same cycle of manufacture. See, 21 CFR Part 210, FDA, Guidance for Industry cGMP for Phase 1 Investigational Drugs, FDA.

As used herein, the terms “Good Manufacturing Practices” or “GMP,” “Current Good Manufacturing Practices” or “cGMP,” and “Active Pharmaceutical Ingredient” or “API,” follow The International Council for Harmonisation of Technical Requirements for Pharmaceuticals for Human Use (ICH) guidelines, which are a set of guidelines to ensure safe, effective, and high-quality medicines are developed and registered efficiently. These guidelines have been adopted by regulatory authorities throughout the world, for example, “Q7 Good Manufacturing Practice Guidance for Active Pharmaceutical Ingredients,” (hereinafter “Q7”), which are incorporated herein by reference.

As used herein, the term “pharmaceutical grade” refers to an Active Pharmaceutical Ingredient, which is manufactured according to Good Manufacturing Practices (GMP) as defined, for example, in Q7.

As used herein, the terms “precipitate”, “precipitation”, “crystallize” and “crystallization” are used interchangeably to refer to unit operations that generate a solid from a saturated solution. As these terms are used herein, no distinction is made as to whether said solid is amorphous or crystalline, has small particles or large particles, or precipitates rapidly or slowly.

As used herein, the term “antisolvent” refers to a solvent in which a compound or salt thereof is poorly soluble and which induces precipitation or crystallization of the compound or salt thereof from solution.

As used herein, the term “chromatographic purification” refers to the separation of a mixture by passing it in solution or suspension or as a vapor (as in gas chromatography) through a medium in which the individual components of the mixture move at different rates. Some examples include column chromatography, flash column chromatography, high performance liquid chromatography, gas chromatography, affinity chromatography, supercritical fluid chromatography, and ion-exchange chromatography, among others known to persons skilled in the art.

As used herein, the term “ambient temperature” refers to the temperature of the immediate surroundings (e.g., room temperature or a reaction environment, e.g., about 20° C. to about 25° C.).

As used herein, the term “radical scavenger” refers to a chemical substance added to a mixture in order to reduce, remove or diminish the reactivity of molecules or ions having one or more unpaired electrons, i.e., radicals.

As used herein, the term “inert gas” refers to a gas such as nitrogen, argon and helium that lack chemical reactivity.

As used herein, the term “dilution purging” refers to a process in which air is purged from an apparatus such as a chemical reaction vessel by introducing an inert gas into the apparatus while allowing any excess gas to escape through an opening, vent or valve.

As used herein, the term “sparging” refers to a process in which a gas is bubbled through a liquid in order to remove other dissolved gas(es) and/or dissolved volatile liquid(s) from that liquid, for example, applying nitrogen in order to remove dissolved oxygen.

As used herein, the term “vacuum purge” refers to a process in which air is purged from an apparatus such as a chemical reaction vessel by extracting the air with a vacuum pump and then introducing an inert gas to the evacuated apparatus. As used herein, the term “vacuum purge cycle(s)” refers to the repetition of the vacuum purge process until the desired concentration of undesired gas, such as oxygen or water vapor, is reached.

As used herein, abbreviations for methods, techniques, limits, units, dimensionless quantities, and measured or calculated values may include High-Performance Liquid Chromatography (HPLC), Ultra Performance Liquid Chromatography (UPLC), Thin Layer Chromatography (TLC), Supercritical Fluid Chromatography (SCF), Capillary Electrophoresis (CE), Ultraviolet Absorbance Detection (UV), Gas Chromatography (GC), Mass Spectrometry (MS), Liquid Chromatography-Mass Spectrometry (LC-MS), Gas Chromatography-Mass Spectrometry (GC-MS), Nuclear Magnetic Resonance Spectroscopy (NMR), Fourier Transform Infrared Spectroscopy (FTIR), Good Manufacturing Practices (GMP), Current Good Manufacturing Practices (cGMP), Parts Per Million (ppm), Retention Time (RT), Relative Retention Time (RRT), Mass-To-Charge Ratio (m/z), Not More Than (NMT), and Not Less Than (NLT).

As used herein, % purity level and % impurity level refer to area %, unless indicated otherwise.

As used herein, the term “bromocriptine” refers to (6aR,9R)-5-bromo-N-((2R,5S,10aS,10bS)-10b-hydroxy-5-isobutyl-2-isopropyl-3,6-dioxooctahydro-2H-oxazolo[3,2-a]pyrrolo[2,1-c]pyrazin-2-yl)-7-methyl-4,6,6a,7,8,9-hexahydroindolo[4,3-fg]quinoline-9-carboxamide.

As used herein, the terms “2-bromolysergic acid diethylamide” and “2-Br-LSD” are used interchangeably and refer to (6aR,9R)-5-bromo-N,N-diethyl-7-methyl-4,6,6a, 7,8,9-hexahydroindolo[4,3-fg]quinoline-9-carboxamide.

As used herein, the terms “iso-2-bromolysergic acid diethylamide” and “iso-2-Br-LSD” are used interchangeably and refer to (6aR,9S)-5-bromo-N,N-diethyl-7-methyl-4,6,6a, 7,8,9-hexahydroindolo[4,3-fg]quinoline-9-carboxamide.

As used herein, the term “lysergic acid” refers to (6aR,9R)-7-methyl-4,6,6a, 7,8,9-hexahydroindolo[4,3-fg]quinoline-9-carboxylic acid.

As used herein, the term “iso-lysergic acid” refers to (6aR,9S)-7-methyl-4,6,6a, 7,8,9-hexahydroindolo[4,3-fg]quinoline-9-carboxylic acid.

As used herein, the term “2-bromolysergic acid” refers to (6aR,9R)-5-bromo-7-methyl-4,6,6a, 7,8,9-hexahydroindolo[4,3-fg]quinoline-9-carboxylic acid.

As used herein, the term “iso-2-bromolysergic acid” refers to (6aR,9S)-5-bromo-7-methyl-4,6,6a, 7,8,9-hexahydroindolo[4,3-fg]quinoline-9-carboxylic acid.

As used herein, the terms “lysergic acid diethylamide” and “LSD” are used interchangeably and refer to (6aR,9R)—N,N-diethyl-7-methyl-4,6,6a,7,8,9-hexahydroindolo[4,3-fg]quinoline-9-carboxamide.

As used herein, the terms “iso-lysergic acid diethylamide” and “iso-LSD” are used interchangeably and refer to (6aR,9S)—N,N-diethyl-7-methyl-4,6,6a,7,8,9-hexahydroindolo[4,3-fg]quinoline-9-carboxamide.

As used herein, the term “2,12-dibromo-lysergic acid” refers to (6aR,9R)-1,5-dibromo-7-methyl-4,6,6a, 7,8,9-hexahydroindolo[4,3-fg]quinoline-9-carboxylic acid.

As used herein, the term “2,13-dibromo-lysergic acid” refers to (6aR,9R)-2,5-dibromo-7-methyl-4,6,6a, 7,8,9-hexahydroindolo[4,3-fg]quinoline-9-carboxylic acid.

As used herein, the term “2,14-dibromo-lysergic acid” refers to (6aR,9R)-3,5-dibromo-7-methyl-4,6,6a,7,8,9-hexahydroindolo[4,3-fg]quinoline-9-carboxylic acid.

As used herein, the term “2,12,13-tribromo-lysergic acid” refers to (6aR,9R)-1,2,5-tribromo-7-methyl-4,6,6a, 7,8,9-hexahydroindolo[4,3-fg]quinoline-9-carboxylic acid.

As used herein, the term “2,12,14-tribromo-lysergic acid” refers to (6aR,9R)-1,3,5-tribromo-7-methyl-4,6,6a, 7,8,9-hexahydroindolo[4,3-fg]quinoline-9-carboxylic acid.

As used herein, the term “2,13,14-tribromo-lysergic acid” refers to (6aR,9R)-2,3,5-tribromo-7-methyl-4,6,6a, 7,8,9-hexahydroindolo[4,3-fg]quinoline-9-carboxylic acid.

As used herein, the terms “2,12-dibromo-lysergic acid diethylamide” and “2,12-dibromo-LSD” are used interchangeably and refer to (6aR,9R)-1,5-dibromo-N,N-diethyl-7-methyl-4,6,6a, 7,8,9-hexahydroindolo[4,3-fg]quinoline-9-carboxamide.

As used herein, the terms “2,13-dibromo-lysergic acid diethylamide” and “2,13-dibromo-LSD” are used interchangeably and refer to (6aR,9R)-2,5-dibromo-N,N-diethyl-7-methyl-4,6,6a, 7,8,9-hexahydroindolo[4,3-fg]quinoline-9-carboxamide.

As used herein, the terms “2,14-dibromo-lysergic acid diethylamide” and “2,14-dibromo-LSD” are used interchangeably and refer to (6aR,9R)-3,5-dibromo-N,N-diethyl-7-methyl-4,6,6a, 7,8,9-hexahydroindolo[4,3-fg]quinoline-9-carboxamide.

As used herein, the terms “2,12,13-tribromo-lysergic acid diethylamide” and “2,12,13-tribromo-LSD” are used interchangeably and refer to (6aR,9R)-1,2,5-tribromo-N,N-diethyl-7-methyl-4,6,6a, 7,8,9-hexahydroindolo[4,3-fg]quinoline-9-carboxamide.

As used herein, the terms “2,12,14-tribromo-lysergic acid diethylamide” and “2,12,14-tribromo-LSD” are used interchangeably and refer to (6aR,9R)-1,3,5-tribromo-N,N-diethyl-7-methyl-4,6,6a, 7,8,9-hexahydroindolo[4,3-fg]quinoline-9-carboxamide.

As used herein, the terms “2,13,14-tribromo-lysergic acid diethylamide” and “2,13,14-tribromo-LSD” are used interchangeably and refer to (6aR,9R)-2,3,5-tribromo-N,N-diethyl-7-methyl-4,6,6a, 7,8,9-hexahydroindolo[4,3-fg]quinoline-9-carboxamide.

As used herein, the term “dimeric dicarboxylic acid impurity” refers to undesirable 2-bromolysergic acid dimeric species tentatively identified as one or more of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid.

The structure of 2-Br-LSD is as follows:

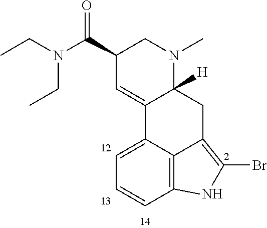 [SMILES: CCN(CC)C(=O)[C@@H]1C=C2c3cccc4[nH]c(Br)c(c34)C[C@H]2N(C)C1]

The structure of iso-2-Br-LSD is as follows:

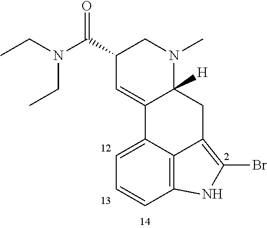 [SMILES: CCN(CC)C(=O)[C@H]1C=C2c3cccc4[nH]c(Br)c(c34)C[C@H]2N(C)C1]

A main advantage of the methods of the present disclosure lies in the relative low levels of impurities, particularly LSD, iso-2-Br-LSD, dimeric species and di- and tri-bromo species, that have plagued known synthetic methods. The disclosed methods produce 2-Br-LSD in relatively high yield and at a high purity. The 2-Br-LSD is substantially free of impurities, particularly LSD, iso-2-Br-LSD, dimeric impurities and di- and tri-bromo species (having one bromine at the desired 2-position and the other bromine(s) on the phenyl ring of the indole moiety at positions 12 and/or 13 and/or 14 (ergoline numbering)).

A first aspect of the present disclosure is directed to a method of synthesizing pharmaceutical grade 2-Br-LSD, or a pharmaceutically acceptable salt thereof, comprising: (a) filtering a mixture comprising 2-Br-LSD and a solvent; (b) allowing the 2-Br-LSD to precipitate from the filtered mixture; (c) collecting the 2-Br-LSD by filtration; (d) analyzing the collected 2-Br-LSD of (c) for the presence of iso-2-Br-LSD; and (e1) if a batch of the collected 2-Br-LSD of (c) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or (e2) if a batch of the collected 2-Br-LSD of (c) fails to meet the pre-set specification for iso-2-Br-LSD, then purifying the collected 2-Br-LSD and repeating (d) and (e), or discarding the collected 2-Br-LSD.

In some embodiments, the 2-Br-LSD may be present substantially in a solution, and filtration of the solution is performed to remove undesired impurities. In some embodiments, the 2-Br-LSD may be allowed to precipitate or crystallize from the filtered mixture, or filtrate, using standard precipitation or crystallization techniques. In some embodiments, precipitation or crystallization may be induced by allowing the filtrate to cool. In some embodiments, precipitation or crystallization may be induced by allowing the filtrate to cool from a temperature above about 20° C. to about 25° C. to about 20° C. to about 25° C. In some embodiments, precipitation or crystallization may be induced by cooling the filtrate to a temperature below about 20° C. to about 25° C. In some embodiments, precipitation or crystallization may be induced by allowing the filtrate to cool to about 0-5° C. In some embodiments, precipitation or crystallization may be induced by slow evaporation of the filtrate. In some embodiments, precipitation or crystallization may be induced by the addition of an antisolvent to the filtrate. In some embodiments, the antisolvent is water. In some embodiments, the antisolvent is water in an amount that does not exceed the maximum solubility of water in the filtrate. In some embodiments, the antisolvent is water in an amount less than about 3.5%. In some embodiments, the antisolvent is water in an amount of about 2 molar equivalents relative to 2-Br-LSD. In some embodiments, precipitation or crystallization may be induced by allowing the vapor of an antisolvent to diffuse into the filtrate.

The analysis of purity and impurity levels may be conducted by HPLC using UV detection (HPLC/UV) at 240 nm under the conditions described in Example 1, HPLC Method 1A, and are provided as area %.

HPLC Method 1A includes the following: System: Agilent 1100/1200 series liquid chromatograph or equivalent; Mobile Phase A: acetonitrile:water (5:95)+trifluoroacetic acid 0.05%; Mobile Phase B: acetonitrile:water (95:5)+trifluoroacetic acid 0.05%; Injection Volume: 5 μL; Flow: 1 mL/min; Column: Kinetex® 2.6 μm XB-C18 100 Å, 100×4.6 mm; Column Temperature: 35° C.; Autosampler Temperature: 4° C.; Detection: UV 240 nm; Sample preparation: Weigh about 25 mg of powder and dilute to 100 mL with Mobile Phase A:Mobile Phase B 60:40.

### Gradient:

| Time (minutes) | % A | % B |
|---|---|---|
| Start | 100.0 | 0.0 |
| 3.00 | 100.0 | 0.0 |
| 15.00 | 20.0 | 80.0 |
| 20.00 | 20.0 | 80.0 |
| 23.00 | 100.0 | 0.0 |

A number of methods for purifying the collected 2-Br-LSD of (c) in (e2) may be employed, for example, extraction, trituration, crystallization, fractional crystallization, recrystallization, or chromatography. In some embodiments, the further purifying is performed in the absence of chromatographic purification. In some embodiments, the further purifying is performed by (a4) suspending the collected 2-Br-LSD of (c) in ethyl acetate or isopropyl acetate, (b4) filtering the suspension, (c4) heating the filtrate to about 30-70° C., (d4) adding water in an amount less than about 3.5%, (e4) allowing the mixture to cool, and (f4) collecting the product by filtration. In some embodiments, the further purifying is performed by (a5) suspending the collected 2-Br-LSD of (c) in ethyl acetate, (b5) filtering the suspension, (c5) heating the filtrate to about 45-50° C., (d5) adding water in an amount of about 2 molar equivalents relative to 2-Br-LSD, (e5) allowing the mixture to cool, and (f5) collecting the product by filtration.

In some embodiments, the pre-set specification for iso-2-Br-LSD is not more than 2%. In some embodiments, the pre-set specification for iso-2-Br-LSD is not more than 0.4%. In some embodiments, the pre-set specification further comprises at least one of the following pre-set specifications: not more than 0.05% of lysergic acid diethylamide (LSD); not more than 0.5% of iso-LSD; not more than 0.15% each of di-bromo-LSD species comprising 2,12-dibromo-LSD, 2,13-dibromo-LSD, and 2,14-dibromo-LSD; not more than 0.15% each of tri-bromo-LSD species comprising 2,12,13-tribromo-LSD, 2,12,14-tribromo-LSD, and 2,13,14-tribromo-LSD; and not more than 1% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR,9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′, N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

In some embodiments, the pre-set specification further comprises at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, not more than 0.15% each of tri-bromo-lysergic acid species comprising 2,12,13-tribromo-LSD, 2,12,14-tribromo-LSD, and 2,13,14-tribromo-LSD, and not more than 0.15% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR, 9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

In some embodiments, the solvent of (a) comprises an organic solvent. In some embodiments, the organic solvent comprises one or more of isopropyl acetate, ethyl acetate, tert-butyl methyl ether, toluene, cyclopropyl methyl ether, butyl acetate, isobutyl acetate, methyl acetate, propyl acetate, methyl ethyl ketone, methyl isobutyl ketone, acetone, and acetonitrile. In some embodiments, the organic solvent comprises isopropyl acetate, ethyl acetate, tert-butyl methyl ether, toluene, or cyclopropyl methyl ether. In some embodiments, the organic solvent comprises isopropyl acetate or ethyl acetate.

In some embodiments, the solvent of (a) further comprises water in an amount that does not exceed the maximum solubility of water in the organic solvent. In some embodiments, the amount of water used in (a) is less than about 3.5%. In some embodiments, an antisolvent is added to the filtered mixture of (a). In some embodiments, water is added to the filtered mixture of (a) in an amount of less than about 3.5%.

In some embodiments, (a) is performed at or below about 20° C. to about 25° C.; the filtered mixture from (a) is heated to ensure complete dissolution prior to the addition of water; and (b) is performed at or below about 20° C. to about 25° C. In some embodiments, (a) is performed at or below about 20° C. to about 25° C.; the filtered mixture from (a) is heated to about 30-50° C. to ensure complete dissolution prior to the addition of water; and (b) is performed at or below about 20° C. to about 25° C.

In some embodiments, (a) is performed at about 20° C. to about 25° C.; the filtered mixture from (a) is heated to about 45-50° C. to ensure complete dissolution prior to the addition of water; and (b) is performed at about 20° C. to about 25° C.

In some embodiments, (a) is performed at a temperature above about 20° C. to about 25° C. and (b) is performed at or below about 20° C. to about 25° C. In some embodiments, (a) is performed at about 25-60° C. In some embodiments, (a) is performed at about 30-50° C.

In some embodiments, (a) is performed at about 45-50° C. In some embodiments, (b) is performed at 0-5° C.

In some embodiments, the 2-Br-LSD of (a) is the reaction product of 2-bromolysergic acid, diethylamine and an amide coupling reagent. In some embodiments, 2-bromolysergic acid is converted into pharmaceutical grade 2-Br-LSD, or a pharmaceutically acceptable salt thereof, in the absence of chromatographic purification. In some embodiments, the reaction is conducted in the presence of an inert solvent. In some embodiments, the inert solvent is an organic solvent.

Representative examples of organic solvents that may be suitable for use in the production of 2-Br-LSD from 2-bromolysergic acid include hydrocarbon solvents, including those containing oxygen, a halogenated solvent, and combinations (mixtures of two or more) thereof. In some embodiments, the solvent is tetrahydrofuran (THF), acetonitrile, dichloromethane, ethyl acetate, isopropyl acetate, toluene, or methyltetrahydrofuran or a combination of two or more thereof. In some embodiments, the solvent is tetrahydrofuran (THF). In some embodiments, the solvent is dichloromethane. In some embodiments, the solvent is acetonitrile. In some embodiments, the solvent includes acetonitrile and dichloromethane.

Representative examples of amide coupling reagents that may be useful in the practice of the present disclosure include carbamates, carbazates, dicyclohexylcarbodiimide (DCC), benzotriazol-1-yloxytripyrrolidinophosphonium hexafluorophosphate (PyBOP), 2-(1H-benzotriazole-1-yl)-1,1,3,3-tetramethylaminium tetrafluoroborate (TBTU), 2-(1H-benzotriazol-1-yl)-1,1,3,3 tetramethyluronium hexafluorophosphate (hexafluorophosphate benzotriazole tetramethyl uronium, or HBTU), 1-[bis(dimethylamino)methylene]-1H-1,2,3-triazolo[4,5-b]pyridinium 3-oxide hexafluorophosphate (hexafluorophosphate azabenzotriazole tetramethyl uronium, or HATU), 1, l′-carbonyldiimidazole (CDI), propylphosphonic anhydride (T3P), N-(3-dimethylaminopropyl)-N′-ethylcarbodiimide (EDC) with or without hydroxybenzotriazole (HOBT), pivaloyl chloride, and acetic anhydride. In some embodiments, the amide coupling reagent is T3P. In some embodiments, the amide coupling reagent is HBTU.

In some embodiments, the 2-Br-LSD of (a) is the reaction product of 2-bromolysergic acid, diethylamine, an amide coupling reagent and a base. In some embodiments, the 2-Br-LSD of (a) is the reaction product of 2-bromolysergic acid, diethylamine, an amide coupling reagent and an acyl transfer promoter. In some embodiments, the 2-Br-LSD of (a) is the reaction product of 2-bromolysergic acid, diethylamine, an amide coupling reagent, a base and an acyl transfer promoter. Representative examples of bases that may be useful in the practice of the present disclosure include triethylamine, N-methylpyrrolidine, N-methylmorpholine (NMM), and 1,4-diazo[2.2.2]octane (DABCO). Representative examples of acyl transfer promoters that may be useful in the practice of the present disclosure include pyridine, N-methylimidazole (NMI), and 4-(dimethylamino)pyridine (DMAP).

In some embodiments, about 1 to about 5 molar equivalents of the amide coupling agent are used for each molar equivalent of 2-bromolysergic acid. In some embodiments, about 1.2 to about 2.5 molar equivalents of the amide coupling agent are used for each molar equivalent of 2-bromolysergic acid. In some embodiments, about 1.1 to about 1.4 molar equivalents of the amide coupling agent are used for each molar equivalent of 2-bromolysergic acid. In some embodiments, about 1.8 to about 2.2 molar equivalents of the amide coupling agent are used for each molar equivalent of 2-bromolysergic acid.

In some embodiments, about 1 to about 10 molar equivalents of diethylamine are used for each molar equivalent of 2-bromolysergic acid. In some embodiments, about 4 to about 6 molar equivalents of diethylamine are used for each molar equivalent of 2-bromolysergic acid.

In some embodiments, about 1 to about 10 molar equivalents of a base are used for each molar equivalent of 2-bromolysergic acid.

In some embodiments, about 0.1 to about 10 molar equivalents of an acyl transfer promoter are used for each molar equivalent of 2-bromolysergic acid.

In some embodiments, about 1 to about 100 mL of organic solvent is used per g of 2-bromolysergic acid. In some embodiments, about 10 mL of organic solvent is used per g of 2-bromolysergic acid.

In some embodiments, the amide coupling is conducted at a temperature between about −20° C. and about 35° C. In some embodiments, the amide coupling is conducted at a temperature between about 0° C. and about 25° C. In some embodiments, the amide coupling is conducted at a temperature between about 0° C. and about 5° C. In some embodiments, the amide coupling is conducted at a temperature between about 15° C. and about 25° C.

In some embodiments, the amide coupling reaction is conducted for about 15 min to about 24 h. In some embodiments, the amide coupling reaction is conducted for about 30 min to about 2 h. In some embodiments, the amide coupling reaction is conducted until in-process analysis shows complete consumption of 2-bromolysergic acid. In some embodiments, in-process analysis is performed by HPLC. In some embodiments, in-process analysis is performed by HPLC at a detection wavelength of about 240 nm. In some embodiments, in-process analysis is performed by TLC. If in-process analysis indicates that substantial 2-bromolysergic acid remains, additional diethylamine may be added to drive the reaction to completion.

In some embodiments, the amide coupling comprises an aqueous workup. In some embodiments, the aqueous workup comprises washing with an aqueous base. Representative examples of aqueous bases that may be useful include potassium carbonate, potassium bicarbonate, sodium carbonate, sodium bicarbonate, sodium hydroxide, and potassium hydroxide. In some embodiments, the aqueous base is aqueous potassium carbonate.

In some embodiments directed to a method of synthesizing pharmaceutical grade 2-Br-LSD, or a pharmaceutically acceptable salt thereof, the mixture of (a) is prepared by (a1) hydrolyzing bromocriptine to form 2-bromolysergic acid, wherein (a1) is conducted in an environment in which radical survival is inhibited, followed by (a2) contacting 2-bromolysergic acid with diethylamine and an amide coupling reagent, to form 2-Br-LSD, followed by (a3) adding the solvent used to prepare the mixture of (a). In some embodiments, (a1) and (a2) are conducted in the absence of chromatographic purification.

Bromocriptine is a semi-synthetic derivative of the ergot alkaloid ergocriptine. Bromocriptine is an approved drug that is used in the treatment of a variety of disorders that result from excessive prolactin or growth hormone secretion from the pituitary gland, as well as type 2 diabetes and Parkinson's disease. An advantage of the present methods that entail use of bromocriptine is that bromocriptine is not a controlled substance. It is readily available from generic active pharmaceutical ingredient (API) manufacturers in multikilogram quantities. In addition, bromocriptine manufactured according to current Good Manufacturing Practices (cGMP) that is highly purified and essentially free of di- and tri-bromo impurities (individual unidentified impurities <0.1%) is less expensive on a molar basis than lysergic acid which has been previously used to manufacture 2-Br-LSD.

Initial attempts at the hydrolysis of bromocriptine to lysergic acid using 4-6 equivalents of KOH in methanol at reflux gave a dark brown, gummy solid that was not readily converted to pure 2-bromo-LSD (data not shown). Various bases such as KOH, NaOH, and LiOH; stoichiometric ratios; solvents such as methanol, ethanol, water, and tetrahydrofuran; temperatures; reaction times; workup procedures and purification methods were tried. Phase transfer conditions, including KOH/tetrabutylammonium bisulphate in dichloromethane/water or dioxane/water, were also investigated. The products from many of these experiments contained a major impurity (up to 34% by HPLC/UV) that did not purge in the next step. In fact, subjecting 2-bromolysergic acid to the same reaction conditions, e.g., aqueous KOH in MeOH at 65° C., gave this impurity as the main product. This impurity was isolated and identified as a dimer with the formula C₃₂H₂₈Br₂N₄O₄.

Applicant has discovered that conducting (a1) in an environment in which radical survival is inhibited substantially reduces the formation of undesirable 2-bromolysergic acid dimeric species tentatively identified as one or more of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, which do not purge in (a2).

In some embodiments, (a1) is conducted in an environment in which radical survival is inhibited. In some embodiments, (a1) is conducted substantially in the absence of oxygen. By the term “substantially free of oxygen,” is meant not more than 100 ppm. In some embodiments, the oxygen in the headspace above the reaction mixture is not more than 100 ppm. In some embodiments, the oxygen in the headspace is not more than 10 ppm. In some embodiments, the oxygen in the headspace is not more than 5 ppm. In some embodiments, the oxygen in the headspace is not more than 1 ppm. In some embodiments, the oxygen in the headspace is not more than 0.5 ppm. In some embodiments, the oxygen in the headspace is not more than 0.2 ppm. In some embodiments, the oxygen dissolved in the reaction mixture is not more than 100 ppm. In some embodiments, the oxygen dissolved in the reaction mixture is not more than 10 ppm. In some embodiments, the oxygen dissolved in the reaction mixture is not more than 1 ppm. By way of illustration, Example 4 describes an inventive embodiment wherein the hydrolysis was carried out in which oxygen was excluded (see, Example 4). The process afforded 2-bromolysergic acid in 94.3% area purity by HPLC/UV, with only 0.33% area of the dimeric impurity.

In some embodiments, (a1) is conducted in the presence of a radical scavenger. In some embodiments, the radical scavenger is sodium dithionite.

In some embodiments, (a1) is conducted substantially in the absence of light.

In some embodiments, the atmosphere substantially free of oxygen is achieved by vacuum-purging, which comprises extracting the air with a vacuum pump and then introducing an inert gas to the evacuated apparatus. In some embodiments, at least three vacuum-purge cycles are performed. In some embodiments, at least five vacuum-purge cycles are performed. In some embodiments, at least ten vacuum-purge cycles are performed. In some embodiments, the vacuum-purge cycles are applied after all of the reactants have been mixed. In some embodiments, the reactants are stirred while vacuum-purge cycles are applied. In some embodiments, the atmosphere substantially free of oxygen is achieved by sparging the reactants with an inert gas. In some embodiments, the sparging in (a1) occurs after all of the reactants have been mixed. In some embodiments, the reactants are stirred while sparging is performed. In some embodiments, sparging in (a1) is continued for the duration of the hydrolysis reaction until the reaction is quenched or workup is commenced. In some embodiments, the atmosphere substantially free of oxygen is achieved by dilution purging of the headspace in the reactor with an inert gas. In some embodiments, the reactants are stirred while dilution purging is performed. In some embodiments, a flow of inert gas over the reaction mixture is maintained for the duration of the hydrolysis reaction until the reaction is quenched or workup is commenced.

In some embodiments, following the purging of oxygen by one of the above methods, the reaction vessel is sealed and a positive pressure is maintained with inert gas. In some embodiments, the positive pressure is about 1 bar. In some embodiments, a headspace oxygen monitor is used to determine when the headspace is substantially free of oxygen. In some embodiments, a dissolved oxygen probe is used to determine when the reaction mixture is substantially free of oxygen. In some embodiments, the inert gas is nitrogen. In some embodiments, the inert gas is argon. In some embodiments, the inert gas comprises not more than 5 ppm oxygen. In some embodiments, the inert gas comprises not more than 1 ppm oxygen. In some embodiments, the inert gas comprises not more than 0.5 ppm oxygen. In some embodiments, the inert gas comprises not more than 0.2 ppm oxygen.

In some embodiments, (a1) comprises contacting bromocriptine with a base in the presence of water. In some embodiments, (a1) comprises contacting bromocriptine with a base in the presence of water and an organic solvent. In some embodiments, (a1) comprises contacting bromocriptine with a base in the presence of water, an organic solvent and a phase transfer agent. In some embodiments, (a1) comprises contacting bromocriptine with an acid. In some embodiments, (a1) comprises contacting bromocriptine with an acid in the presence of water. In some embodiments, (a1) comprises contacting bromocriptine with an acid in the presence of an organic solvent. In some embodiments, (a1) comprises contacting bromocriptine with an acid in the presence of water and an organic solvent.

Representative examples of organic solvents that may be suitable for use in the reaction include hydrocarbon solvents, including those containing one or more of the following elements: oxygen, nitrogen, sulfur, chlorine, fluorine. In some embodiments, a mixture of organic solvents is used. In some embodiments, the organic solvent is methanol (MeOH). Other organic solvents include acetic acid, acetone, acetonitrile, 1-butanol, 2-butanol, tert-butanol, chlorobenzene, chloroform, 1,2-dichloroethene, dichloromethane, 1,2-dimethoxyethane, dimethylsulfoxide, 1,4-dioxane, ethanol, 2-ethoxyethanol, ethylene glycol, formic acid, 2-methoxyethanol, nitromethane, 1-propanol, isopropanol, sulfolane, tetrahydrofuran, and 1,1,2-trichloroethene.

In some embodiments, the base is a strong base. In some embodiments, the strong base is an alkali metal base or a hydrate thereof. In some embodiments, the strong base is potassium hydroxide (KOH). In some embodiments, the strong base is lithium hydroxide (LiOH) or lithium hydroxide monohydrate (LiOH·H₂O). In some embodiments, the strong base is sodium hydroxide (NaOH). In some embodiments, the strong base is an alkaline earth metal base or a hydrate thereof. In some embodiments, the strong base is calcium hydroxide (Ca(OH)₂). In some embodiments, the strong base is barium hydroxide (Ba(OH)₂). In some embodiments, the strong base is an alkali metal alkoxide. In some embodiments, the strong base is sodium methoxide (NaOMe). In some embodiments, the strong base is lithium methoxide (LiOMe). In some embodiments, the strong base is sodium ethoxide (NaOEt). In some embodiments, the strong base is potassium tert-butoxide (KOt-Bu). In some embodiments, the strong base is ammonium hydroxide (NH₄OH). In some embodiments, the strong base is a tetraalkylammonium hydroxide or a hydrate thereof. In some embodiments, the strong base is tetrapropylammonium hydroxide. In some embodiments, the strong base is tetrabutylammonium hydroxide. Other tetraalkylammonium hydroxides include tetramethylammonium hydroxide pentahydrate, triethylmethylammonium hydroxide, tetraethylammonium hydroxide, choline hydroxide, hexamethonium hydroxide, hexadecyltrimethylammonium hydroxide, tetrapentylammonium hydroxide, tetrahexylammonium hydroxide, and trimethylphenylammonium hydroxide. In some embodiments, the base is potassium carbonate.

In some embodiments, the acid is a strong acid or a hydrate thereof. In some embodiments, the strong acid is sulfuric acid (H₂SO₄). In some embodiments, the strong acid is hydrochloric acid (HCl). Other strong acids include hydrobromic acid (HBr), hydrofluoric acid (HF), p-toluenesulfonic acid, methanesulfonic acid, trifluoromethanesulfonic acid, trifluoroacetic acid, trichloroacetic acid, and perchloric acid.

In some embodiments, the phase transfer agent is a quaternary ammonium salt or a hydrate thereof. In some embodiments, the quaternary ammonium salt is tetrabutylammonium bisulfate (Bu₄NHSO₄), where Bu=n-butyl. Other phase transfer agents include tetrabutylammonium bromide (Bu₄NBr), benzyltributylammonium bromide (Bu₃(Bn)NBr, where Bn=benzyl), tetraethylammonium chloride (Et₄NCl, where Et=ethyl), tetrabutylammonium hydroxide (Bu₄NOH), Adogen 464 [Me(C₈₋₁₀H₁₇₋₂₁)₃NCl], Aliquat 336 [Me(C₈H₁₇)₃NCl], and benzyltriethylammonium chloride (Bn(Et)₃NCl), among others. Phase transfer reagents and methods for their use are described, for example, in Ikunaka, M. Organic Process Research & Development 12, 698-709 (2008), and references cited therein.

In some embodiments, about 0.1 to about 10 molar equivalents of the strong base, e.g., KOH, are used for each molar equivalent of bromocriptine. In some embodiments, about 5 to about 6 molar equivalents are used for each molar equivalent of bromocriptine. In some embodiments, about 5 molar equivalents are used for each molar equivalent of bromocriptine.

In some embodiments, about 1 to about 20 mL of organic solvent is used per g of bromocriptine. In some embodiments, about 1 to about 20 ml of water is used per g of bromocriptine. In some embodiments, about 3 mL of organic solvent and about 2 mL of water are used per g of bromocriptine.

In some embodiments, (a1) further comprises isolating the 2-bromolysergic acid as a solid prior to (a2). In some embodiments, the isolating comprises adjustment of the pH, which facilitates the formation of the intermediate, 2-bromolysergic acid, in solid form. In some embodiments, the isolating comprises dilution of the mixture with a solvent and adjustment of the pH. In some embodiments, the dilution solvent is water. In some embodiments, the pH adjustment is conducted using an acid such as hydrochloric acid (HCl). In some embodiments, the pH adjustment is to a pH value below about 6. In some embodiments, the pH is adjusted to about 4.7. In some embodiments, the isolating comprises filtering, washing, and drying the solid 2-bromolysergic acid.

The order of mixing the reactants in (a1) is not critical. In some embodiments, (a1) comprises forming a reaction mixture containing bromocriptine, a solvent, a strong base, and water. In some embodiments, the reaction mixture is formed by adding the strong base and water to a reaction vessel containing bromocriptine and a solvent.

Selection of other reaction parameters such as temperature and time are within the level of skill in the art. In some embodiments, (a1) is conducted at an internal temperature between about 45° C. and about 70° C. In some embodiments, (a1) is conducted at an internal temperature of about 65° C. to 70° C. In some embodiments, (a1) is conducted for about 30 min to about 48 hours. In some embodiments, (a1) is conducted for about 5 to about 24 hours. In some embodiments, (a1) is conducted for about 15 to about 24 hours.

In some embodiments, (a)-(e2) are performed in the absence of chromatographic purification. In some embodiments, the entire manufacturing process from bromocriptine to a batch of pharmaceutical grade 2-Br-LSD, or a pharmaceutically acceptable salt thereof, is performed in the absence of chromatographic purification.

The 2-Br-LSD, or a pharmaceutically acceptable salt thereof, may be isolated as a solid in accordance with standard techniques. In some embodiments, the isolating comprises collecting the solid via filtration. In some embodiments, the solid may be crystalline or amorphous, and if the former, have one or more polymorphic forms. In some embodiments, the crystalline solid may exist in one or more polymorphic forms. In some embodiments, the solid comprises crystalline solid 2-Br-LSD.

A related aspect of the present disclosure provides a method of synthesizing 2-bromolysergic acid according to Good Manufacturing Practice (GMP) as an Active Pharmaceutical Ingredient (API) or as an intermediate for API manufacturing, according to the methods described herein that entail hydrolyzing bromocriptine in an environment in which radical survival is inhibited (e.g., wherein in some embodiments, (a1) is conducted substantially in the absence of oxygen).

A further aspect of the present disclosure provides a batch of 2-bromolysergic acid, or a pharmaceutically acceptable salt thereof, prepared according to the methods of the disclosure.

In some embodiments, the batch of 2-bromolysergic acid contains not more than 0.15% each of di-bromo-lysergic acid species comprising 2,12-dibromo-lysergic acid, 2,13-dibromo-lysergic acid, and 2,14-dibromo-lysergic acid, and tri-bromo-lysergic acid species comprising 2,12,13-tribromo-lysergic acid, 2,12,14-tribromo-lysergic acid, and 2,13,14-tribromo-lysergic acid; contains not more than 0.05% of lysergic acid; contains not more than 1% each of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid; contains not more than 6% of iso-2-bromolysergic acid; and contains not less than 90% of 2-bromolysergic acid.

In some embodiments, the batch of 2-bromolysergic acid contains not more than 0.1% each of 2,12-dibromo-lysergic acid, 2,13-dibromo-lysergic acid, and 2,14-dibromo-lysergic acid, and not more than 0.05% each of 2,12,13-tribromo-lysergic acid, 2,12,14-tribromo-lysergic acid, and 2,13,14-tribromo-lysergic acid. In some embodiments, the batch of 2-bromolysergic acid contains not more than 0.05% each of 2,12-dibromo-lysergic acid, 2,13-dibromo-lysergic acid, 2,14-dibromo-lysergic acid, 2,12,13-tribromo-lysergic acid, 2,12,14-tribromo-lysergic acid, and 2,13,14-tribromo-lysergic acid. In some embodiments, the batch of 2-bromolysergic acid contains not more than 0.5% each of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid.

In some embodiments, the total amount of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid is not more than 0.5%. In some embodiments, the batch of 2-bromolysergic acid contains not more than 4% of iso-2-bromolysergic acid. In some embodiments, the batch of 2-bromolysergic acid contains not less than 94% of 2-bromolysergic acid. In some embodiments, the batch of 2-bromolysergic acid contains not less than 96% of 2-bromolysergic acid. In some embodiments, the batch of 2-bromolysergic acid is manufactured according to GMP as an intermediate for API manufacturing or as an API.

Another aspect of the present disclosure provides a batch of pharmaceutical grade 2-Br-LSD, or a pharmaceutically acceptable salt thereof, prepared according to the methods of the disclosure.

The 2-Br-LSD may be salified to produce a pharmaceutically acceptable salt by methods known in the art. The term “pharmaceutically acceptable salt” refers to a product obtained by reaction of the 2-Br-LSD with a suitable acid.

The purified solid 2-Br-LSD may also be solvated (i.e., with pharmaceutically acceptable solvents such as water, ethanol, and the like, for example, as a hydrate (e.g., mono-hydrate, di-hydrate, etc.)).

Another aspect of the present disclosure is directed to a pharmaceutical composition that comprises a therapeutically effective amount of pharmaceutical grade 2-Br-LSD, e.g., made by the disclosed process, or a pharmaceutically acceptable salt thereof, and a pharmaceutically acceptable carrier. In some embodiments, the 2-Br-LSD has a purity level of at least 95% area, as measured by HPLC/UV. In some embodiments, the 2-Br-LSD has a purity level of at least 97% area, as measured by HPLC/UV. In some embodiments, the 2-Br-LSD has a purity level of at least 98% area, as measured by HPLC/UV. In some embodiments, the 2-Br-LSD has a purity level of at least 99% area, as measured by HPLC/UV.

The term “pharmaceutically acceptable carrier,” as known in the art, refers to a pharmaceutically acceptable material, composition, or vehicle, suitable for administering pharmaceutical grade 2-Br-LSD, or a pharmaceutically acceptable salt thereof, to mammals. Suitable carriers may include, for example, liquids (both aqueous and non-aqueous alike, and combinations thereof), solids, encapsulating materials, and combinations thereof (e.g., semi-solids). A carrier is “pharmaceutically acceptable” in the sense of being physiologically inert, compatible with the other ingredients of the formulation, and not injurious to the subject or patient. Depending on the type of formulation, the composition may further include one or more pharmaceutically acceptable excipients.

Broadly, pharmaceutical grade 2-Br-LSD and pharmaceutically acceptable salts thereof of the present disclosure may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999 Marchcel Dekker, New York).

In some embodiments, 2-Br-LSD and pharmaceutically acceptable salts thereof are formulated for oral or intravenous administration (e.g., systemic intravenous injection).

In some embodiments, the 2-Br-LSD and pharmaceutically acceptable salts thereof may be formulated into solid compositions (e.g., powders, tablets, dispersible granules, capsules, cachets, and suppositories). In some embodiments, the 2-Br-LSD and pharmaceutically acceptable salts thereof may be formulated into liquid compositions (e.g., solutions in which the 2-Br-LSD and pharmaceutically acceptable salts thereof is dissolved, suspensions in which solid particles of the 2-Br-LSD and pharmaceutically acceptable salts thereof are dispersed, emulsions, and solutions containing liposomes, micelles, or nanoparticles, syrups and elixirs). The 2-Br-LSD and pharmaceutically acceptable salts thereof may also be formulated for rapid, intermediate or extended release.

Such compositions may be prepared in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999 Marchcel Dekker, New York).

The 2-Br-LSD can be used for a variety of pharmacological uses. In some embodiments, the 2-Br-LSD may be used to treat disorders associated with pain, e.g., cephalic pain, migraine headaches, vascular headaches, cluster headaches (see, e.g., U.S. Pat. No. 8,415,371, and to reduce stroke damage (see, e.g., U.S. Pat. No. 4,524,072).

These and other aspects of the present disclosure will be further appreciated upon consideration of the following Examples, which are intended to illustrate certain particular embodiments of the disclosure but are not intended to limit its scope, as defined by the claims.

### Specific Embodiments

Representative embodiments of the present disclosure are set forth in the following paragraphs.

Paragraph 1. A method of synthesizing pharmaceutical grade 2-bromolysergic acid diethylamide (2-Br-LSD), or a pharmaceutically acceptable salt thereof, comprising:
a. filtering a mixture comprising 2-Br-LSD and a solvent;
b. allowing the 2-Br-LSD to precipitate from the filtered mixture;
c. collecting the 2-Br-LSD by filtration;
d. analyzing the collected 2-Br-LSD of (c) for the presence of iso-2-Br-LSD; and
e. (1) if a batch of the collected 2-Br-LSD of (c) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or
e. (2) if a batch of the collected 2-Br-LSD of (c) fails to meet the pre-set specification for iso-2-Br-LSD, then purifying the collected 2-Br-LSD and repeating (d) and (e), or discarding the collected 2-Br-LSD.

Paragraph 2. The method of paragraph 1, wherein the pre-set specification for iso-2-Br-LSD is not more than 2%.

Paragraph 3. The method of paragraph 1 or 2, wherein the pre-set specification for iso-2-Br-LSD is not more than 0.4%.

Paragraph 4. The method of any one of paragraphs 1-3, wherein the pre-set specification further comprises at least one of the following pre-set specifications: not more than 0.05% of lysergic acid diethylamide, not more than 0.5% of iso-lysergic acid diethylamide, not more than 0.15% each of di-bromo-lysergic acid diethylamide species comprising 2,12-dibromo-lysergic acid diethylamide, 2,13-dibromo-lysergic acid diethylamide, and 2,14-dibromo-lysergic acid diethylamide, not more than 0.15% each of tri-bromo-lysergic acid diethylamide species comprising 2,12,13-tribromo-lysergic acid diethylamide, 2,12,14-tribromo-lysergic acid diethylamide, and 2,13,14-tribromo-lysergic acid diethylamide, and not more than 1% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR,9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

Paragraph 5. The method of any one of paragraphs 1-4, wherein the pre-set specification further comprises at least one of the following pre-set specifications: not more than 0.05% of lysergic acid diethylamide, not more than 0.05% of iso-lysergic acid diethylamide, not more than 0.15% each of di-bromo-lysergic acid diethylamide species comprising 2,12-dibromo-lysergic acid diethylamide, 2,13-dibromo-lysergic acid diethylamide, and 2,14-dibromo-lysergic acid diethylamide, not more than 0.15% each of tri-bromo-lysergic acid diethylamide species comprising 2,12,13-tribromo-lysergic acid diethylamide, 2,12,14-tribromo-lysergic acid diethylamide, and 2,13,14-tribromo-lysergic acid diethylamide, and not more than 0.15% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR,9R,9'S)-5,5′-dibromo-N9,N9,N9′, N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

Paragraph 6. The method of any one of paragraphs 1-5, wherein (a)-(e2) are performed in the absence of chromatographic purification.

Paragraph 7. The method of any one of paragraphs 1-6, wherein the solvent of (a) comprises an organic solvent.

Paragraph 8. The method of paragraph 7, wherein the organic solvent comprises isopropyl acetate or ethyl acetate.

Paragraph 9. The method of paragraph 7, wherein the solvent of (a) further comprises water in an amount of less than about 3.5%.

Paragraph 10. The method of paragraph 7, wherein water is added to the filtered mixture of (a) in an amount of less than about 3.5%.

Paragraph 11. The method of paragraph 10, wherein (a) is performed at or below about 20° C. to about 25° C., the filtered mixture from (a) is heated to ensure complete dissolution prior to the addition of water, and (c) is performed at or below about 20° C. to about 25° C.

Paragraph 12. The method of any one of paragraphs 1-10, wherein (a) is performed at a temperature above about 20° C. to about 25° C. and (c) is performed at or below about 20° C. to about 25° C.

Paragraph 13. The method of paragraph 12, wherein (a) is performed at about 30-50° C.

Paragraph 14. The method of any one of paragraphs 1-13, wherein the 2-Br-LSD of (a) is the reaction product of 2-bromolysergic acid, diethylamine and an amide coupling reagent.

Paragraph 15. The method of paragraph 14, wherein the 2-bromolysergic acid is converted into pharmaceutical grade 2-Br-LSD, or a pharmaceutically acceptable salt thereof, in the absence of chromatographic purification.

Paragraph 16. The method of paragraph 14 or 15, wherein the reaction is conducted in the presence of an inert solvent.

Paragraph 17. The method of paragraph 16, wherein the inert solvent is tetrahydrofuran (THF).

Paragraph 18. The method of any one of paragraphs 14-17, wherein the amide coupling reagent is propylphosphonic anhydride (T3P), 2-(1H-benzotriazol-1-yl)-1,1,3,3-tetramethyluronium hexafluorophosphate (Hexafluorophosphate Benzotriazole Tetramethyl Uronium, or HBTU), 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium, or HATU), 1,1′-Carbonyldiimidazole (CDI), or acetic anhydride.

Paragraph 19. The method of any one of paragraphs 14-18, wherein the amide coupling reagent is T3P.

Paragraph 20. The method of any one of paragraphs 14-18, wherein the amide coupling reagent is HBTU.

Paragraph 21. The method of any one of paragraphs 1-20, wherein the mixture of (a) is prepared by (a1) hydrolyzing bromocriptine to form 2-bromolysergic acid, wherein (a1) is conducted in an environment in which radical survival is inhibited, followed by (a2) contacting 2-bromolysergic acid with diethylamine and an amide coupling reagent, to form 2-Br-LSD, followed by (a3) adding the solvent used to prepare the mixture of (a).

Paragraph 22. The method of paragraph 21, wherein (a1) and (a2) are conducted in the absence of chromatographic purification.

Paragraph 23. The method of any one of paragraphs 21-22, wherein (a1) is conducted substantially in the absence of oxygen.

Paragraph 24. The method of any one of paragraphs 21-22, wherein (a1) is conducted in the presence of a radical scavenger.

Paragraph 25. The method of any one of paragraphs 21-24, wherein (a1) comprises contacting bromocriptine with a base in the presence of water and an organic solvent.

Paragraph 26. The method of paragraph 25, wherein the base is potassium hydroxide.

Paragraph 27. The method of paragraph 25 or 26, wherein the organic solvent is methanol.

Paragraph 28. A method of synthesizing 2-bromolysergic acid according to GMP as an Active Pharmaceutical Ingredient (API) or as an intermediate for API manufacturing, comprising hydrolyzing bromocriptine in an environment in which radical survival is inhibited.

Paragraph 29. The method of paragraph 28, wherein the hydrolyzing is conducted substantially in the absence of oxygen.

Paragraph 30. The method of paragraph 28 or 29, wherein the hydrolyzing is conducted in the presence of a radical scavenger.

Paragraph 31. The method of any one of paragraphs 28-30, wherein the hydrolyzing comprises contacting bromocriptine with a base in the presence of water and an organic solvent.

Paragraph 32. The method of paragraph 31, wherein the base is potassium hydroxide.

Paragraph 33. The method of paragraph 31 or 32, wherein the organic solvent is methanol.

Paragraph 34. A batch of pharmaceutical grade 2-Br-LSD, or a pharmaceutically acceptable salt thereof, prepared according to the method of any one of paragraphs 1-27.

Paragraph 35. A batch of 2-bromolysergic acid, or a pharmaceutically acceptable salt thereof, prepared according to the method of any one of paragraphs 28-33.

Paragraph 36. A batch of 2-bromolysergic acid that:
a. contains not more than 0.15% each of di-bromo-lysergic acid species comprising 2,12-dibromo-lysergic acid, 2,13-dibromo-lysergic acid, and 2,14-dibromo-lysergic acid, and tri-bromo-lysergic acid species comprising 2,12,13-tribromo-lysergic acid, 2,12,14-tribromo-lysergic acid, and 2,13,14-tribromo-lysergic acid;
b. contains not more than 0.05% of lysergic acid;
c. contains not more than 1% each of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid;
d. contains not more than 6% of iso-2-bromolysergic acid; and
e. contains not less than 90% of 2-bromolysergic acid.

Paragraph 37. The batch of paragraph 36, that contains not more than 0.1% each of 2,12-dibromo-lysergic acid, 2,13-dibromo-lysergic acid, and 2,14-dibromo-lysergic acid, and not more than 0.05% each of 2,12,13-tribromo-lysergic acid, 2,12,14-tribromo-lysergic acid, and 2,13,14-tribromo-lysergic acid.

Paragraph 38. The batch of paragraph 36 or 37, that contains not more than 0.05% each of 2,12-dibromo-lysergic acid, 2,13-dibromo-lysergic acid, 2,14-dibromo-lysergic acid, 2,12,13-tribromo-lysergic acid, 2,12,14-tribromo-lysergic acid, and 2,13,14-tribromo-lysergic acid.

Paragraph 39. The batch of any one of paragraphs 36-38, that contains not more than 0.5% each of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid.

Paragraph 40. The batch of any one of paragraphs 36-39, wherein total amount of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid is not more than 0.5%.

Paragraph 41. The batch of any one of paragraphs 36-40, that contains not more than 4% of iso-2-bromolysergic acid.

Paragraph 42. The batch of any one of paragraphs 36-41, that contains not less than 94% of 2-bromolysergic acid.

Paragraph 43. The batch of any one of paragraphs 36-42, that contains not less than 96% of 2-bromolysergic acid.

Paragraph 44. The batch of any one of paragraphs 36-43 that is manufactured according to GMP as an intermediate for API manufacturing or as an API.

Paragraph 45. A pharmaceutical composition, comprising the batch of paragraph 34, or a portion thereof, and a pharmaceutically acceptable carrier.

### EXAMPLES

### Example 1: HPLC Methods

### HPLC Method 1:

Mobile Phase A: acetonitrile:water (5:95)+trifluoroacetic acid 0.05%
Mobile Phase B: acetonitrile:water (95:5)+trifluoroacetic acid 0.05%
Injection Volume: 5 μL
Flow: 1 mL/min
Column: Kinetex® 2.6 μm XB-C18 100 Å, 100×4.6 mm
Column Temperature: 35° C.
Autosampler Temperature: 4° C.
Detection: UV 240 nm

### Gradient:

| Time (minutes) | % A | % B |
|---|---|---|
| Start | 100.0 | 0.0 |
| 3.00 | 100.0 | 0.0 |
| 15.00 | 20.0 | 80.0 |
| 20.00 | 20.0 | 80.0 |
| 23.00 | 100.0 | 0.0 |

HPLC Method 1A: Same as Method 1. Sample preparation: Weigh about 25 mg of powder and dilute to 100 mL with Mobile Phase A:Mobile Phase B 60:40.

HPLC Method 1B: Same as Method 1. Sample preparation: Weigh about 160 mg of reaction mixture and dilute to 50 mL with water:acetonitrile 40:60.

HPLC Method 1C: Same as Method 1. Sample preparation: Weigh about 20 mg of powder and dilute to 100 mL with water:acetonitrile 40:60.

HPLC Method 1D: Same as Method 1. Sample preparation: Weigh about 160 mg of reaction mixture and dilute to 50 mL with Mobile Phase A:Mobile Phase B 40:60.

### HPLC Method 2:

Mobile Phase A: acetonitrile:water (5:95)+trifluoroacetic acid 0.05%
Mobile Phase B: acetonitrile:water (95:5)+trifluoroacetic acid 0.05%
Diluent: Mobile Phase A:Mobile Phase B (60:40)
Injection Volume: 0.5 μL
Column: Acquity UPLC HSS T3 1.8 μm, 100×2.1 mm
Column Temperature: 25° C.
Autosampler Temperature: 4° C.
Detection: UV 240 nm
Sample preparation: Weigh about 125 mg of powder and dilute to 50 mL with Mobile Phase
A:Mobile Phase B (60:40).

### Gradient:

| Time (minutes) | % A | % B | Flow (mL/min) |
|---|---|---|---|
| Start | 90.0 | 10.0 | 0.45 |
| 20.00 | 90.0 | 10.0 | 0.45 |
| 21.00 | 45.0 | 55.0 | 0.40 |
| 28.00 | 45.0 | 55.0 | 0.40 |
| 29.00 | 90.0 | 10.0 | 0.45 |
| 35.00 | 90.0 | 10.0 | 0.45 |

### Example 2: Synthesis of 2-Bromolysergic Acid Diethylamide

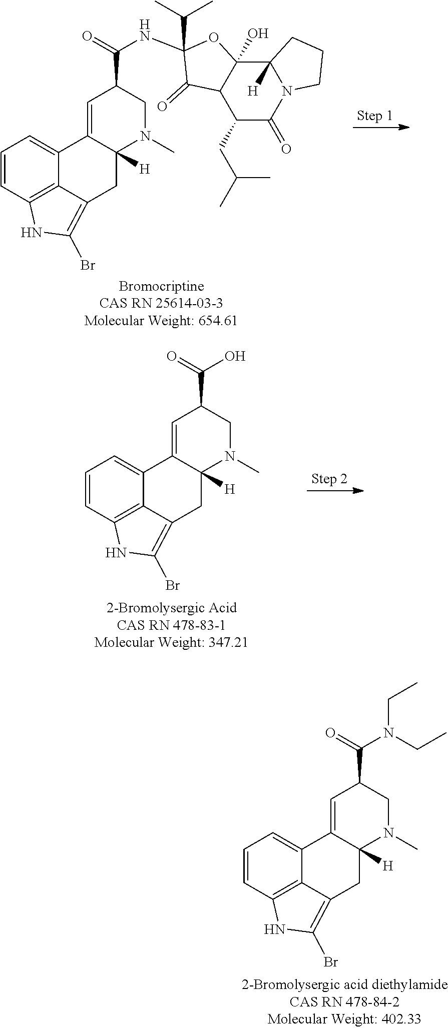

All processing steps were conducted under an atmosphere of nitrogen, unless indicated otherwise.

### Step 1: 2-Bromolysergic Acid

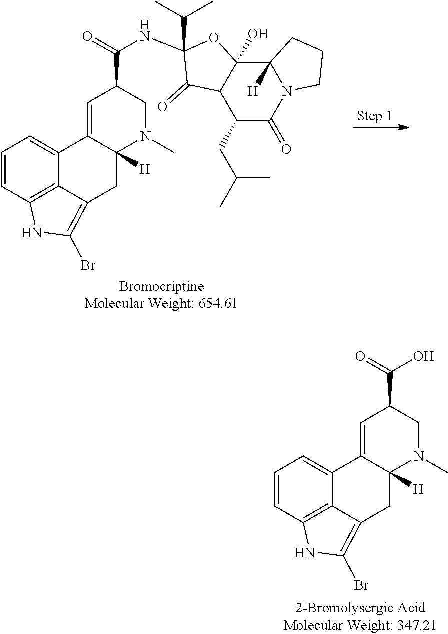

LIST OF STARTING MATERIALS AND REAGENTS:

| Material | Amount | | MW | mol | Ratio | |
|---|---|---|---|---|---|---|
| bromocriptine | 1.089 | kg | 654.61 | 1.66 | 1.0 | equivalents |
| MeOH | 3.27 | L | | | 3 | volumes |
| KOH | 0.467 | kg | 56.11 | 8.32 | 5.0 | equivalents |
| H₂O | 2.178 | L | | | 2 | volumes |
| H₂O | 5.445 | L | | | 5 | volumes |
| HCl 37% | 0.59 | L | | | 0.54 | volumes |
| H₂O | 1.198 | L | | | 1.1 | volumes |
| H₂O | 1.089 | L | | | 1 | volume |
| H₂O | 1.089 | L | | | 1 | volume |

Bromocriptine (1.089 kg) was charged to a reactor through the hatch, followed by methanol (3.27 L), resulting in a suspension. A solution of H₂O (2.178 L) and KOH (0.467 Kg), prepared in advance, was added through the hatch to the stirred suspension of bromocriptine in methanol at room temperature. Ten vacuum-purge cycles (vacuum-nitrogen) were performed, and the reaction mixture was maintained under a nitrogen atmosphere protected from light. The jacket temperature was set at 80±1° C. and the reaction mixture was stirred for 21 hours. The internal temperature rose to 70° C. After 21 hours, in-process analysis by HPLC Method 1B showed 89.8% 2-bromolysergic acid (specification: >86%). The reaction mixture was cooled to 50±2° C. and H₂O (5.445 L) was charged. Then the reaction mixture was cooled to 30° C. A solution of HCl 37% (0.59 L) in H₂O (1.198 L), prepared in advance, was added slowly, dropwise in not less than 4 hours to the reaction mixture through a dropping funnel while maintaining the internal temperature at 30±1° C., until pH=4.7 was achieved. The addition took about 4 hours. A solid was formed. The suspension was stirred for a further 1 hour and allowed to cool spontaneously to 25±2° C. The solid product was isolated on a Büchner filter, washed with water (2×1.089 L) and dried in a vacuum oven at 60° C. for 15 hours to afford a light brown solid, 410.5 g (71% yield). Analysis by HPLC Method 1C indicated 97.1%2-bromolysergic acid (as an unresolved mixture of diastereomers) and 0.62% of the dimeric dicarboxylic acid impurity tentatively identified as one or more of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid. Analysis by HPLC Method 2 revealed the ratio of 2-bromolysergic acid to iso-2-bromolysergic acid to be 96.5:3.5. Moisture content was 0.48% as measured by Karl Fischer titration.

### Step 2: 2-Bromolysergic Acid Diethylamide

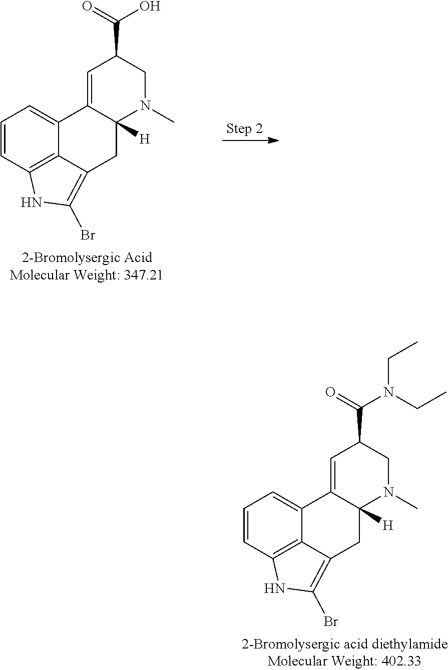 [SMILES: CN1C[C@H](C(=O)O)C=C2c3cccc4[nH]c(Br)c(c34)C[C@H]21 ; CCN(CC)C(=O)[C@@H]1C=C2c3cccc4[nH]c(Br)c(c34)C[C@H]2N(C)C1]

LIST OF STARTING MATERIALS AND REAGENTS:

| Material | Amount | | MW | mmol | Ratio | |
|---|---|---|---|---|---|---|
| 2-bromolysergic acid | 50.0 | g | 347.21 | 144 | 1.0 | equivalents |
| dichloromethane | 500 | mL | | | 10 | volumes |
| diethylamine | 89.4 mL | | 73.14 | 864.0 | 6.0 | equivalents |
| | (d = 0.707 g/mL) | | | | | |
| propylphosphonic anhydride | 183.3 | g | 318.18 | 288.0 | 2.0 | equivalents |
| (T3P) solution | | | | | | |
| (50% in CH₃CN) | | | | | | |
| K₂CO₃, 10% sol. in H₂O | 2 × 100 | mL | | | 2 × 2 | volumes |
| H₂O | 100 | mL | | | 2 | volumes |
| ethyl acetate | 1350 | mL | | | 27 | volumes |
| H₂O | 13 | mL | 18.02 | 722.2 | 5.0 | equivalents |
| ethyl acetate | 2 × 12.5 | mL | | | 2 × 0.25 | volumes |

In a suitable reactor 2-bromolysergic acid (50.0 g) was charged, followed by dichloromethane (500 mL), resulting in a suspension. Diethylamine (89.4 mL) was added dropwise to this stirred suspension at room temperature, resulting in a dark solution. The mixture was cooled to 0-5° C. The T3P 50% solution in CH3CN was added dropwise at such a rate that the temperature remained below 5° C. The addition took about 45 minutes. The reaction mixture was then stirred at 0-5° C. for 1 hour, at which time in-process analysis by HPLC Method 1D showed that 0.02% 2-bromolysergic acid remained (specification: <0.3%) and the ratio of 2-Br-LSD to iso-2-Br-LSD was 85.6:14.4.

The reaction mixture was extracted twice with 100 mL of a 10% aqueous K₂CO₃ and once with 100 mL of water. The organic phase was concentrated under vacuum while maintaining the internal temperature at 20-35° C. until 2 volumes (100 mL) of residue was obtained. Ethyl acetate (400 mL) was added, and the mixture was concentrated under vacuum while maintaining the internal temperature at 20-35° C. until 2 volumes (100 mL) of residue was obtained. This addition of ethyl acetate (400 mL) followed by concentration to 2 volumes (100 mL) was repeated twice more. The residue was diluted to 5 volumes (250 mL) by the addition of ethyl acetate (150 mL). The mixture was stirred for 4-16 hours at 22±2° C. and a suspension was obtained. The solid was filtered off. The solid was dried under vacuum at 45° C. overnight and analyzed for information only (typically 6 g, ratio 2-Br-LSD to iso-2-Br-LSD 3:97 by HPLC Method 1D), then discarded. The ratio of 2-Br-LSD to iso-2-Br-LSD in the supernatant was typically 96:4. The filtrate was transferred to a clean reactor and heated to 45-50° C., whereupon H₂O (13 mL, 5.0 equivalents) was added. The mixture was allowed to cool spontaneously with stirring to room temperature (22° C.) overnight. The product was collected on a Büchner filter, washed with ethyl acetate (2×12.5 mL) and dried in a vacuum oven at 45° C. for 18 hours. The product was obtained as a brown solid, 44.98 g (78.2% yield). Analysis by HPLC Method 1A indicated 97.8%2-Br-LSD and 0.77% iso-2-Br-LSD.

### Purification of 2-Bromolysergic Acid Diethylamide

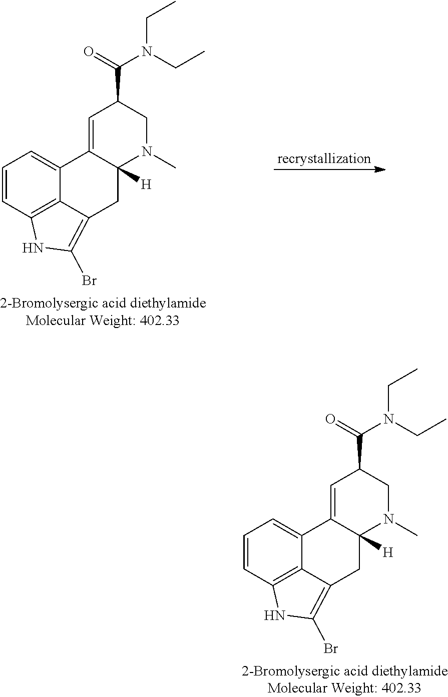 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4[nH]c(Br)c(c34)C[C@H]2N(C)C1]

LIST OF STARTING MATERIALS AND REAGENTS:

| Material | Amount | | MW | mmol | Ratio | |
|---|---|---|---|---|---|---|
| 2-Br-LSD | 206.58 | g | 402.33 | 513.5 | 1.0 | equivalent |
| ethyl acetate | 1033 | mL | | | 5 | volumes |
| H₂O | 18.5 | mL | 18.02 | 1027 | 2.0 | equivalents |
| ethyl acetate | 2 × 41.3 | mL | | | 2 × 0.2 | volumes |

In a suitable reactor, 2-Br-LSD (206.58 g) was charged, followed by ethyl acetate (1033 mL). The mixture was stirred at 20-25° C. overnight. The resulting suspension was filtered. The solid was discarded. The filtrate was charged to a clean reactor. The mixture was heated at 45-50° C. and H₂O (18.5 mL, 2.0 equivalents) was added. The mixture was stirred and allowed to cool spontaneously to room temperature (22° C.) overnight. The product was filtered through a Büchner filter, washed with ethyl acetate (2×41.3 mL) and dried in a vacuum oven at 45° C. for 18 hours. The product was obtained as a brown solid, 151.0 g (73.1% yield). Analysis by HPLC Method 1A indicated 98.08%2-Br-LSD, 0.40% iso-2-Br-LSD and 0.07% dimeric dicarboxylic acid impurity.

### Example 3: Synthesis of 2-Bromolysergic Acid Diethylamide Hemi-L-Tartrate

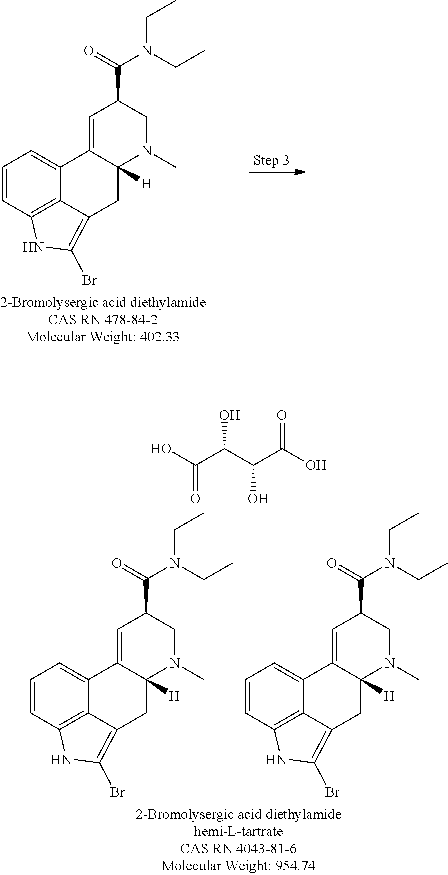

All processing steps were conducted under an atmosphere of nitrogen, unless indicated otherwise.

LIST OF STARTING MATERIALS AND REAGENTS:

| Material | Amount | | MW | mmol | Ratio | |
|---|---|---|---|---|---|---|
| 2-Br-LSD | 9.1 | g | 402.33 | 22.6 | 1.0 | equivalent |
| Isopropanol | 45 | mL | | | 5 | volumes |
| L-tartaric acid | 1.70 | g | 150.09 | 11.3 | 1.0 | equivalent |
| Isopropanol | 71 | mL | | | 7.8 | volumes |
| Isopropanol | 4.5 | mL | | | 0.5 | volumes |
| Isopropanol | 4.5 | mL | | | 0.5 | volumes |

To a suitable reactor was charged 2-Br-LSD (9.1 g, prepared according to Example 2) followed by isopropanol (45 mL), resulting in a dark solution. The mixture was cooled to 15° C. To another vessel was charged L-tartaric acid (1.70 g) followed by isopropanol (71 mL). Upon stirring for 40 minutes at room temperature, the suspended solid dissolved to yield a solution. The solution of L-tartaric acid was added dropwise to the solution of 2-bromolysergic acid at such a rate as to maintain the temperature at 15±2° C. Upon completion of the addition, a suspension formed. The suspension was stirred for 18 hours while being allowed to warm spontaneously to room temperature (22±2° C.). The suspension was filtered through a Büchner filter, washed with isopropanol (2×4.5 mL) and dried in a vacuum oven at 45° C. for 18 hours. The final product was obtained as a light grey solid, 8.1 g (75% yield; 27% overall yield from bromocriptine). Analysis by HPLC Method 1A indicated 99.6%2-Br-LSD and 0.01% iso-2-Br-LSD. The dimeric dicarboxylic acid impurity was not detected. Potentiometric assay indicated 16.3% tartaric acid content (calcd. 15.7% for 2-bromolysergic acid diethylamide hemi-L-tartrate). The structure of the product was demonstrated by HPLC and LCMS comparison with an authentic sample having the following characteristics. FTIR: 1627 cm⁻¹ (amide carbonyl) and 1444 cm⁻¹ (carboxylic acid carbonyl). Electrospray MS: m/z 402.3, 404.3 ([MH]⁺). ¹H and ¹³C NMR, see Tables 1 and 2 (Jeol ECX400 spectrometer, de-DMSO, referenced to 8-2.50 ppm (¹H NMR), δ=39.51 ppm (¹³C NMR)).

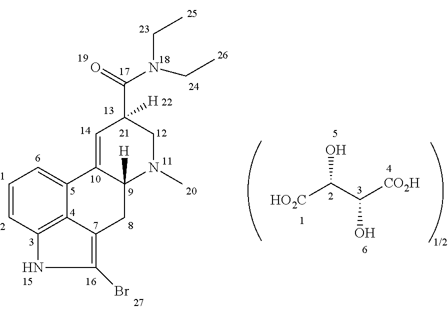

Structure of 2-Bromo-LSD Hemi-L-Tartrate with Atom Numbering

TABLE 1
¹H NMR Table of Assignments

| | Number of | | Coupling | Peak Assignment (atom number as |
|---|---|---|---|---|
| δ/ppm | H | Multiplicity | Constants/Hz | per structure above) |
| 1.05 | 3 | t | 7.0 | 2 × CH₃ (25, 26) |
| 1.17 | 3 | t | 7.0 | |
| 2.44 | 1 | dd | 14.7, 11.0 | 1 × CHH (8) |
| 2.53 | 3 | s | — | 1 × CH₃ (20) |
| 2.69 | 1 | t | 10.8 | 1 × CHH (12) |
| 3.05 | 1 | dd | 11.0, 5.0 | 1 × CHH (12) |
| 3.12-3.21 | 1 | m | — | 1 × CH (21) |
| 3.24-3.37 | 3 | m | — | 2 × CH₂ (23, 24) and |
| 3.44 | 2 | q | 7.0 | 1 × CHH (8) |
| 3.80-3.87 | 1 | m | | CH (22) |
| 4.23 | 1 | s | — | Tartaric acid 2 × CH (2, 3) Hemi Salt |
| 6.28 | 1 | s | — | 1 × CH (14) |
| 7.05-7.17 | 2 | m | — | 3 × CH (1, 6, 2) |
| 11.51 | 1 | s | — | 1 × NH (15) |

TABLE 2
¹³C Table of Assignments

| | Peak Assignment (atom number |
|---|---|
| δ/ppm | as per structure above) |
| 13.08 | 2 × CH₃ (25, 26) |
| 14.83 | |
| 25.85 | CH₂ (8) |
| 36.69 | CH (13) |
| 39.57, 41.58 | 2 × CH₂ (23, 24) |
| 43.09 | CH₃ (20) |
| 55.38 | CH₂ (12) |
| 61.97 | CH (9) |
| 72.07 | Tartaric acid 2 × CH (2, 3) |
| 103.87 | C (7) |
| 108.97 | C (16) |
| 109.35, 112.10, 122.90 | 3 × CH (1, 2, 6) |
| 120.86 | CH (14) |
| 125.71 | C (4) |
| 126.15 | C (5) |
| 133.85 | C (10) |
| 134.22 | C (3) |
| 170.33 | CON (17) |
| 173.53 | Tartaric acid 2 × CO₂H (1, 4) |

### Example 4: Synthesis of 2-Bromolysergic Acid

All processing steps were conducted under an atmosphere of nitrogen, unless indicated otherwise.

2-Bromolysergic acid: In an aluminum pressure reactor, bromocriptine (15.0 g, FW 654.61, 1.0 equiv.) was charged, followed by methanol (45 mL, 3 volumes). In another vessel, water (30 mL, 2 volumes) was charged, followed by potassium hydroxide (KOH) (7.4 g, FW 56.11, 5.76 equiv.). The mixture was stirred until a clear solution was obtained. The solution of KOH in water was added to the suspension of bromocriptine. The batch was sparged with nitrogen and placed under 1 bar of nitrogen pressure in the sealed reactor with a bath temperature of 80° C. for 15 hours. The reaction mixture was cooled to 40° C. and diluted with water (75 mL, 5 volumes). The batch was cooled to 30° C., and a solution of HCl 37% (5.4 mL) in water (9 mL) was added dropwise in around 15 minutes, resulting in a pH value of 8.4. A solution of HCl 37% (4 mL) in water (7 mL) was added dropwise in around 15 minutes, resulting in a pH value of 4.7. A solid formed and the suspension was cooled to 18±2° C. The product was collected on a Büchner filter, washed with water (2×15 mL) and dried in a vacuum oven at 60° C. for 15 hours to afford 6.3 g of pink powder in 79% yield and 94.3% purity by HPLC/UV, with only 0.33% of the dimeric dicarboxylic acid impurity.

### Example 5: Synthesis of 2-Bromolysergic Acid Diethylamide Hydrobromide (Form 1) According to GMP as an Active Pharmaceutical Ingredient

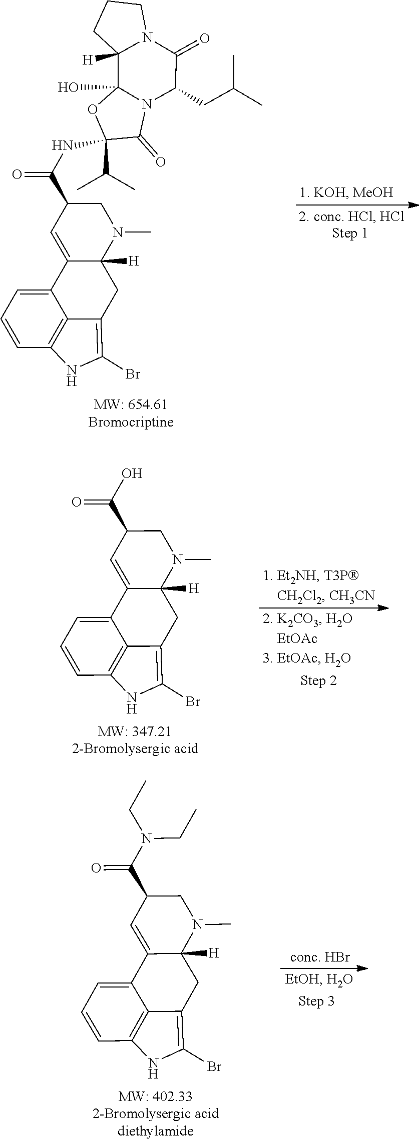 [SMILES: CC(C)C[C@H]1C(=O)N2CCC[C@H]2[C@]2(O)O[C@](NC(=O)[C@@H]3C=C4c5cccc6[nH]c(Br)c(c56)C[C@H]4N(C)C3)(C(C)C)C(=O)N12 ; CN1C[C@H](C(=O)O)C=C2c3cccc4[nH]c(Br)c(c34)C[C@H]21 ; CCN(CC)C(=O)[C@@H]1C=C2c3cccc4[nH]c(Br)c(c34)C[C@H]2N(C)C1]

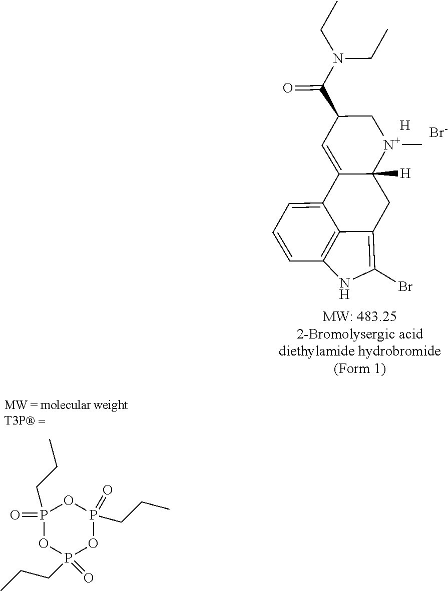

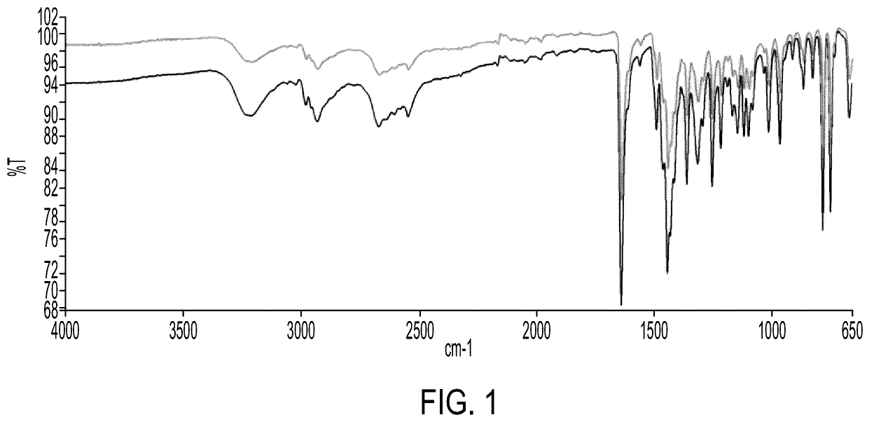

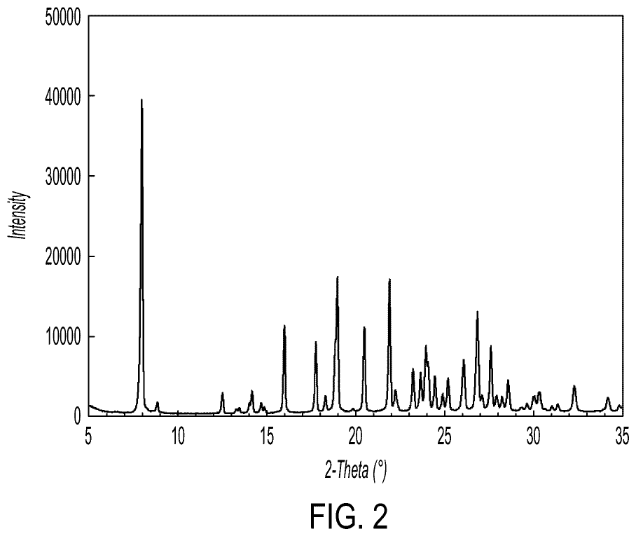

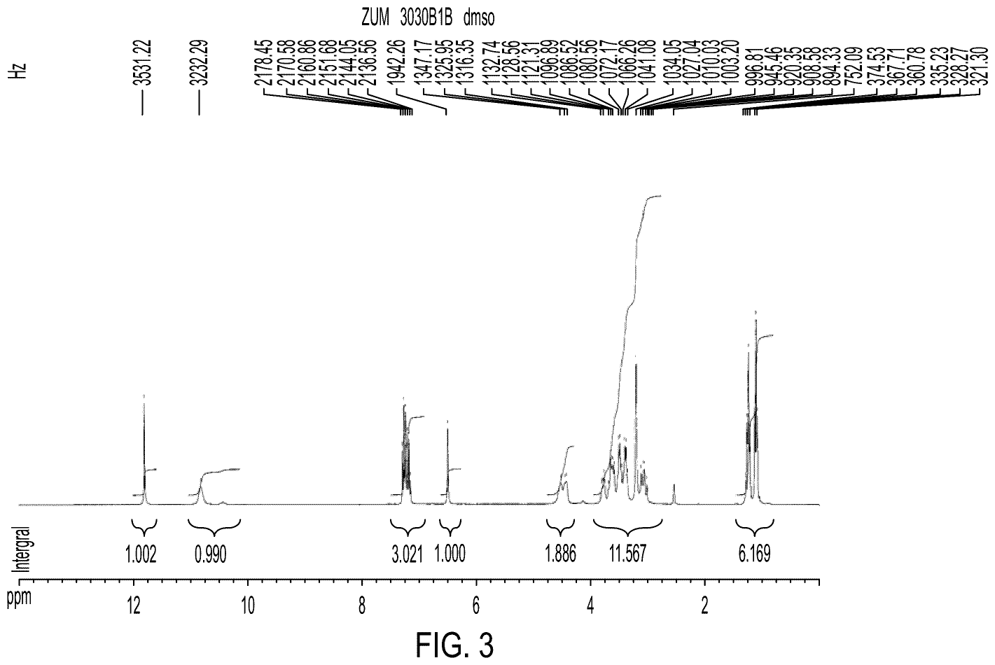

The following process was performed in accordance with GMP appropriate for early-stage clinical use as defined in Q7. All processing steps were conducted under an atmosphere of nitrogen, unless indicated otherwise. The 2-bromolysergic acid diethylamide hydrobromide (Form 1) salt is the subject of Applicant's co-pending application [U.S. Non-Provisional application Ser. No. 18/400,204 (Attorney Ref. No. 58205-00001USNP1), entitled “SALTS OF 2-BROMOLYSERGIC ACID DIETHYLAMIDE”]. Example 2, Methods 1-4, and FIGS. 1-3, 10-11, 19-30 and 46-47 of Applicant's co-pending application describe preparation and characterization of 4 different batches of the 2-bromolysergic acid diethylamide hydrobromide (Form 1) salt. The batch of 2-bromolysergic acid diethylamide hydrobromide (Form 1) salt described in Example 2, Method 3, and illustrated in FIGS. 19-25 of Applicant's co-pending application, is referred to herein as a “reference standard.” Example 6 herein (and FIGS. 3-9 referenced in Example 6) correspond to Example 2, Method 3 and FIGS. 19-25 in Applicant's co-pending application. Where appropriate, the product obtained via the process described in this present Example 5 was compared to the reference standard batch. STEP 3 in this present Example 5 (and FIGS. 1-2 referenced therein), which describes and characterizes production of the 2-bromolysergic acid diethylamide hydrobromide (Form 1) salt from the free base, correspond to Example 2, Method 4 and FIGS. 46-47 of Applicant's co-pending application.

### Step 1: 2-Bromolysergic Acid

A clean, dry reactor was subjected to three vacuum (≤400 mbar)-nitrogen purge cycles. Bromocriptine (12.0 kg) was charged through the hatch, followed by three vacuum (≤400 mbar)-nitrogen purge cycles. Methanol (28.5 kg) was charged through the hatch, followed by three vacuum (≤400 mbar)-nitrogen purge cycles and commencement of agitation. A second reactor was subjected to three vacuum (≤400 mbar)-nitrogen purge cycles, followed by charging of demineralized water (24.0 L) and potassium hydroxide flakes (5.1 kg). Agitation was begun, the temperature was regulated to 23-27° C., and the resulting KOH solution was transferred to the first reactor. Demineralized water (2.4 L) was charged to the second reactor and then transferred to the first reactor. The first reactor was subjected to the maximum possible vacuum (170 mbar), followed by ten vacuum (≤200 mbar)-nitrogen purge cycles. Thereafter, the reaction mixture was maintained under a nitrogen atmosphere protected from light. The reaction mixture was heated to reflux (jacket temperature 78-82° C.; internal temperature 65-71° C.) and stirred for 18 hours. The reaction mixture was cooled to 48-52° C., whereupon in-process analysis by HPLC Method 1B showed 90%2-bromolysergic acid (specification: ≥86%). Demineralized water (60 L) was charged and the reaction mixture was cooled to 30-34° C. A solution of HCl 32% (9.1 kg) in demineralized water (12 L), prepared in advance, was added dropwise in not less than 3 hours to the reaction mixture through a dropping funnel while maintaining the internal temperature at 30-34° C., until pH=4.5-4.9 was achieved. The addition took 3 hours and the final pH was 4.9. A solid was formed. The suspension was cooled to 20-24° C. and stirred for a further 1 hour. The solid product was isolated on a Büchner filter, washed with water (2×12 L) and dried in a vacuum oven at 43-47° C. for not less than 24 hours until in-process controls (IPCs) for appearance (pink to light brown powder or solid) and moisture by Karl Fischer titration (KF; ≤4%) were met to give 4.687 kg of brown powder. Analysis by HPLC Method 1C showed 97.4%2-bromolysergic acid (as an unresolved mixture of diastereomers; specification ≥90%) and 0.06% of the dimeric dicarboxylic acid impurity tentatively identified as one or more of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid. Analysis by HPLC Method 2 revealed the ratio of 2-bromolysergic acid to iso-2-bromolysergic acid to be 94.8:5.2 (specification ≥94:6).

### Step 2: 2-Bromolysergic Acid Diethylamide

A clean, dry reactor was subjected to three vacuum (≤400 mbar)-nitrogen purge cycles. 2-Bromolysergic acid (4.687 kg) was charged, followed by dichloromethane (62.0 kg), followed by three vacuum (≤400 mbar)-nitrogen purge cycles and commencement of agitation. Diethylamine (5.9 kg) was charged, followed by three vacuum (≤400 mbar)-nitrogen purge cycles. The mixture was cooled to an internal temperature of 0-4° C. and T3P 50% solution in CH3CN (17.2 kg) was added at such a rate that the internal temperature remained at 0-4° C. The addition took about 40 minutes. The reaction mixture was then stirred at 0-4° C. for 1 hour, at which time in-process analysis by HPLC Method 1D showed that 0.25%2-bromolysergic acid remained (specification: ≤0.3%).

The reaction mixture was extracted twice with aqueous K₂CO₃ (1.1 kg dissolved in 9.4 kg of demineralized water) and once with demineralized water (9.4 kg). The organic phase was concentrated under vacuum while maintaining the internal temperature at 20-35° C. until 2 volumes (9.4 L) of residue was obtained. Ethyl acetate (34.0 kg) was added and the mixture was concentrated under vacuum while maintaining the internal temperature at 20-35° C. until 2 volumes (9.4 L) of residue was obtained. This addition of ethyl acetate (34.0 kg) followed by concentration to 2 volumes (9.4 L) was repeated twice more. The residue was diluted to 5 volumes (23.6 L) by the addition of ethyl acetate (12.7 kg). The mixture was subjected to three vacuum (≤400 mbar)-nitrogen purge cycles and then stirred for 18 hours at 20-24° C. and a suspension was obtained. The solid was filtered off and discarded. The filtrate was heated to 45-50° C., whereupon demineralized water (1.2 kg) was added. The mixture was cooled in not less than one hour to 20-24° C. and stirred at this temperature for at least 18 hours. The product was collected on a Büchner filter, washed with two approximately 5 kg portions of wet ethyl acetate (prepared by washing 10.0 kg of ethyl acetate with 9.4 L of demineralized water) and dried in a vacuum oven at 43-47° C. for not less than 18 hours until IPCs for appearance (beige or brown powder or solid) and KF (≤2.0%) were met, to afford 3.991 kg of product. Analysis by HPLC Method 1A indicated 82.1%2-Br-LSD, 16.7% iso-2-Br-LSD and dimeric dicarboxylic acid impurity not detected (limit of detection (LOD) 0.02%).

### Purification of 2-Bromolysergic Acid Diethylamide

A clean, dry reactor was subjected to three vacuum (≤400 mbar)-nitrogen purge cycles. 2-Bromo-LSD (3.991 kg) was charged, followed by ethyl acetate (18.0 kg). The mixture was subjected to three vacuum (≤400 mbar)-nitrogen purge cycles and stirred at 20-24° C. for not less than 18 hours. The resulting suspension was filtered and the solid was discarded. The filtrate was heated to 45-50° C., whereupon demineralized water (0.36 L) was added. The mixture was cooled in not less than 1 hour to 20-24° C. and stirred at this temperature for not less than 18 hours. The product was collected on a Büchner filter, washed with two approximately 1.8 kg portions of wet ethyl acetate (prepared by washing 3.6 kg of ethyl acetate with 3.9 L of demineralized water) and dried in a vacuum oven at 43-47° C. for not less than 18 hours until until IPCs for appearance (beige or brown powder or solid), KF (≤1.5%) and purity (2-Br-LSD ≥98.5%; iso-2-Br-LSD≤0.4%) were met, to afford 2.32 kg of light brown powder. Analysis by HPLC Method 1A indicated 99.3%2-Br-LSD, 0.14% iso-2-Br-LSD and dimeric dicarboxylic acid impurity not detected (LOD 0.02%). Although this batch was not directly analyzed for the following potential impurities, it is reasonably inferred from historical data acquired during process development that it would contain not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, not more than 0.15% each of tri-bromo-lysergic acid species comprising 2,12,13-tribromo-LSD, 2,12,14-tribromo-LSD, and 2,13,14-tribromo-LSD, and not more than 0.15% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR, 9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

### Step 3: 2-Bromolysergic Acid Diethylamide Hydrobromide (Form 1)

A clean, dry reactor was subjected to three vacuum (≤400 mbar)-nitrogen purge cycles. 2-Bromo-LSD (2.320 kg) was charged through the hatch, followed by three vacuum (≤400 mbar)-nitrogen purge cycles. A second clean, dry reactor was subjected to three vacuum (≤400 mbar)-nitrogen purge cycles. Denatured ethanol (15.5 kg) and purified water (1.0 L) were charged to the second reactor, followed by three vacuum (≤400 mbar)-nitrogen purge cycles, and this mixture was agitated for not less than 5 minutes. The ethanol-water mixture was transferred to the reactor containing 2-Br-LSD, agitation was begun, and the resulting mixture was heated to 43-47° C., resulting in complete dissolution of the solid. Inside a glove box, denatured ethanol (0.3 kg), purified water (0.02 L) and hydrobromic acid 48% (0.2 kg) were measured out and charged to a dropping funnel. The contents of the dropping funnel were added in not less than 120 minutes to the stirred 2-Br-LSD solution while maintaining an internal temperature of 43-47° C. Stirring was continued at this temperature for not less than 2 hours. Inside the glove box, denatured ethanol (1.3 kg), purified water (0.1 L) and hydrobromic acid 48% (0.8 kg) were measured out and charged to the dropping funnel. The contents of the dropping funnel were added in not less than 180 minutes to the batch while maintaining an internal temperature of 43-47° C. With continuous agitation, the batch was allowed to cool spontaneously to an internal temperature of 20-24° C. and maintained in this range for 18 hours. The batch was cooled to 0-5° C., stirred for 60 minutes, and filtered. Purified water (0.1 L) and denatured ethanol (1.7 kg) were charged to the reactor, and this mixture was cooled to 0-5° C. and used to wash the solid on the filter. The solid was dried in a vacuum oven at 43-47° C. for not less than 18 hours until in-process controls (IPCs) for residual solvents (≤ ICH limits for solvents used in the process) and moisture by Karl Fischer titration (KF; ≤0.5%) were met, to give 2.410 kg of off-white powder.

The product was characterized and released in accordance with GMP for early-stage clinical use as defined in Q7. Selected acceptance specifications and results are presented in Table 3. Where applicable, the results were compared to those of the forementioned reference standard of 2-bromolysergic acid diethylamide hydrobromide (Form 1).

TABLE 3
Selected Release Testing Results for 2-Bromolysergic acid diethylamide
hydrobromide (Form 1) Prepared According to Example 5

| | | |
|---|---|---|
| Test | Specification | Result |

Selected Release Tests

| Appearance | Off-white to light brown powder | Complies | |
|---|---|---|---|
| Identification | | | |
| Infrared spectrum | Conforms with reference spectrum | Complies ^{a} | |
| HPLC retention time ^{b} | Retention time of main peak is ±5% of | Complies | |
| | retention time of reference standard | | |
| HBr content by potentiometric | 15.7-17.7% | 16.8% | |
| titration (% w/w on anhydrous basis) | | | |
| Assay by HPLC (% w/w against | 97.0-02.0% | 99.7% | |
| reference standard, on anhydrous, | | | |
| solvent-free basis) ^{b} | | | |
| Chemical purity by HPLC (% a/a) ^{b} | NLT 98.0% | 99.4% | |
| Related substances by HPLC (% a/a) ^{b} | | | |
| LSD ^{c} | NMT 0.05% | ND ^{d} | |
| RRT 0.99 ^{e} | NMT 0.50% | 0.25% | |
| Iso-2-Br-LSD (RRT 1.16)^{f} | NMT 0.15% | ND ^{g} | |
| Largest single unknown or | NMT 0.15% | RRT 0.86 = 0.11% | |
| unqualified impurity | | RRT 1.17 = 0.05% | |
| Report all impurities greater than | | RRT 1.21 = 0.13% | |
| LOQ (0.05%) | | RRT 1.38 = 0.07% | |
| Total impurities | NMT 2.0% | 0.6% | |
| Residual solvents by HS-GC | | | |
| Methanol | NMT 3000 ppm | 10 | ppm |
| Dichloromethane | NMT 600 ppm | 10 | ppm |
| Acetonitrile | NMT 410 ppm | 4 | ppm |
| Diethylamine | NMT 1000 ppm | 90 | ppm |
| Ethyl acetate | NMT 5000 ppm | 13 | ppm |
| Ethanol | NMT 5000 ppm | 2471 | ppm |
| Acetone | NMT 5000 ppm | 132 | ppm |
| 2-Propanol | NMT 5000 ppm | 115 | ppm |

| | | |
|---|---|---|
| Water content by KF (% w/w) | NMT 0.5% | 0.1% |
| Residue on ignition (% w/w) | NMT 0.5% | 0.0% |
| XRPD | Conforms to Form 1 reference diffractogram | Complies ^{h} |

Additional Tests for Information Only

| | | |
|---|---|---|
| Identification | | |
| ¹H NMR | Report result - compare to reference spectrum | Conforms |
| Mass spectrometry | Report result | [M + H]⁺ 402.6 |

^{a} FIG. 1
^{b} HPLC Method 1D, except that diluent is Mobile Phase A:Mobile Phase B 40:60
^{c} (6aR,9R)-N,N-diethyl-7-methyl-4,6,6a,7,8,9-hexahydroindolo[4,3-fg]quinoline-9-carboxamide
^{d} No peak assignable to LSD was detected by HPLC with UV absorbance detection at 240 nm with LOD 0.02% and ion trap mass detection at m/z 324 ([M + H]⁺)
^{e} Tentatively assigned as (6aR)-5-bromo-N,N-diethyl-7-methyl-4,6,6a,7,8,10a-hexahydroindolo[4,3-fg]quinoline-9-carboxamide
^{f}(6aR,9S)-5-bromo-N,N-diethyl-7-methyl-4,6,6a,7,8,9-hexahydroindolo[4,3-fg]quinoline-9-carboxamide
^{g} LOD 0.02%
^{h} FIG. 2
% w/w = % weight-to-weight;
% a/a = % of total peak area;
NMT = not more than;
NLT = not less than;
RRT = relative retention time;
ND = not detected;
LOD = limit of detection;
HPLC = high performance liquid chromatography;
HS-GC = headspace gas chromatography;
ppm = parts per million;
KF = Karl Fischer moisture titration;
XRPD = x-ray powder diffraction;
NMR = nuclear magnetic resonance spectroscopy

The XRPD pattern for 2-bromolysergic acid diethylamide hydrobromide (Form 1) prepared according to Example 5 was collected using a Bruker D2 X-ray Generator (30 kV, 10 mA, scan interval 3-35°, Step 0.03°, 2 sec/step; flat plate zero-background sample holder, rotation 15 rpm). The results are presented graphically in FIG. 2 and in tabular form in Table 4.

TABLE 4
XRPD Diffraction Peaks for 2-Bromolysergic acid diethylamide
hydrobromide (Form 1) Prepared According to Example 5

| Pos. [°2Th.] | Rel. Int. [%] |
|---|---|
| 8.0 ± 0.2 | 100.0 |
| 8.9 ± 0.2 | 4.3 |
| 12.5 ± 0.2 | 7.2 |
| 14.2 ± 0.2 | 8.2 |
| 14.7 ± 0.2 | 4.2 |
| 16.0 ± 0.2 | 28.7 |
| 17.8 ± 0.2 | 23.1 |
| 18.3 ± 0.2 | 6.5 |
| 19.0 ± 0.2 | 43.0 |
| 20.5 ± 0.2 | 29.2 |
| 21.9 ± 0.2 | 43.1 |
| 22.2 ± 0.2 | 8.4 |
| 23.2 ± 0.2 | 14.9 |
| 23.6 ± 0.2 | 13.6 |
| 23.9 ± 0.2 | 22.2 |
| 24.4 ± 0.2 | 13.1 |
| 24.9 ± 0.2 | 7.1 |
| 25.2 ± 0.2 | 12.0 |
| 26.1 ± 0.2 | 18.2 |
| 26.8 ± 0.2 | 32.6 |
| 27.1 ± 0.2 | 6.6 |
| 27.6 ± 0.2 | 22.7 |
| 27.9 ± 0.2 | 6.4 |
| 28.2 ± 0.2 | 6.2 |
| 28.6 ± 0.2 | 11.7 |
| 30.0 ± 0.2 | 6.1 |
| 30.3 ± 0.2 | 7.5 |
| 31.3 ± 0.2 | 3.8 |
| 32.3 ± 0.2 | 9.4 |
| 34.2 ± 0.2 | 5.4 |
| 36.0 ± 0.2 | 7.0 |
| 38.2 ± 0.2 | 9.3 |

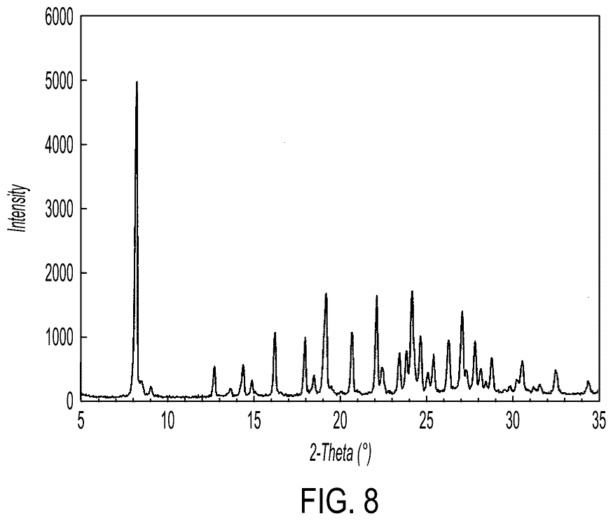

This XRPD pattern is consistent with that of the 2-Br-LSD hydrobromide (Form 1) reference standard prepared according to Example 6, as presented in FIG. 8 and Table 5.

### Example 6: Synthesis of 2-Bromolysergic Acid Diethylamide Hydrobromide (Form 1) Reference Standard

In a suitable reactor, 2-Br-LSD (150.0 g) was charged, followed by denatured ethanol (1275 mL) and water (67.5 mL). The total solvent volume (1342.5 mL) corresponded to 8.95 volumes and an ethanol/water ratio of 95:5. The mixture was heated to 45° C., resulting in complete dissolution. Aqueous (aq.) HBr (2 M) was prepared in a 200 mL volumetric flask by bringing to volume 68.8 g of aq. HBr 47% w/w with a 95:5 mixture of denatured ethanol and water to obtain a concentration of 2 M. The solution was homogenized, and then 37.3 mL of this solution (0.2 equiv, consisting of 12.8 g of aq. HBr 47% w/w, 27.3 mL of denatured ethanol and 1.4 mL of water) was added to the reaction vessel via a dropping funnel over 80 minutes while maintaining an internal temperature of 45° C. At the end of the addition, the mixture was seeded with 0.15 g of 2-bromolysergic acid diethylamide hydrobromide and stirred for two hours at 45° C. Aqueous HBr (2 M) 149.3 mL (0.8 equiv, consisting of 51.4 g of HBr_{aq} 47% w/w, 109.1 mL of denatured ethanol and 5.7 mL of water) was added to the reaction vessel via a dropping funnel over 180 minutes while maintaining an internal temperature of 45° C. The mixture was allowed to spontaneously cool to room temperature (20-25° C.) and stirred overnight. The resulting suspension was then cooled to 0-5° C. and stirred for 60 minutes. The product was collected on a Büchner filter, washed with denatured ethanol/water (95:5, 142.5 mL of ethanol, 7.5 mL of water) and dried in a vacuum oven at 45° C. for 18 hours to give 127.5 g (70.7% molar yield, 85.0% w/w yield).

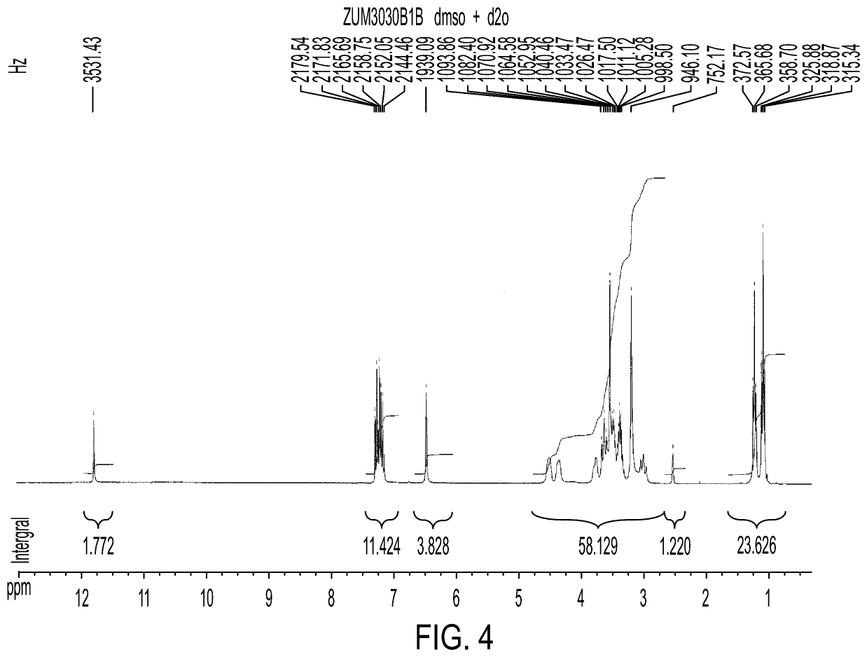

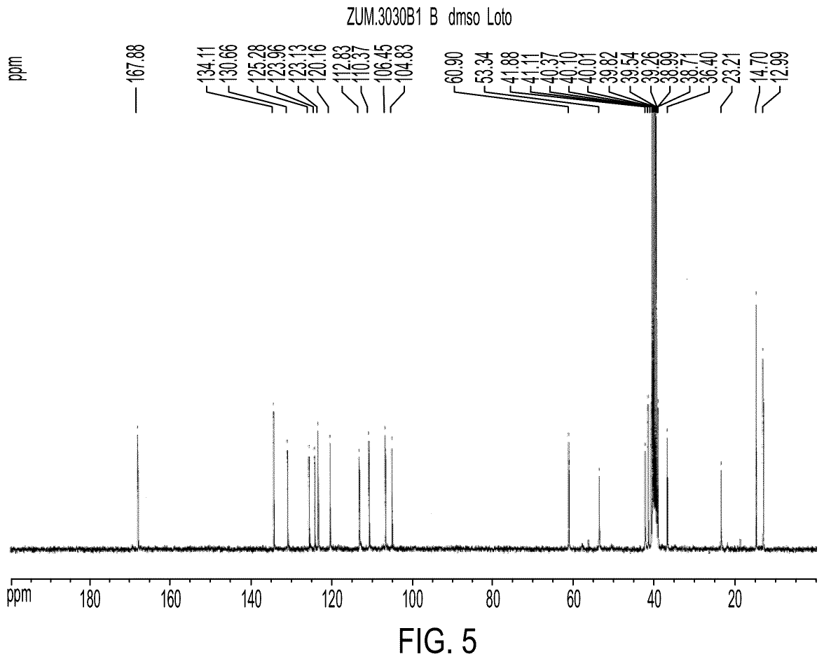

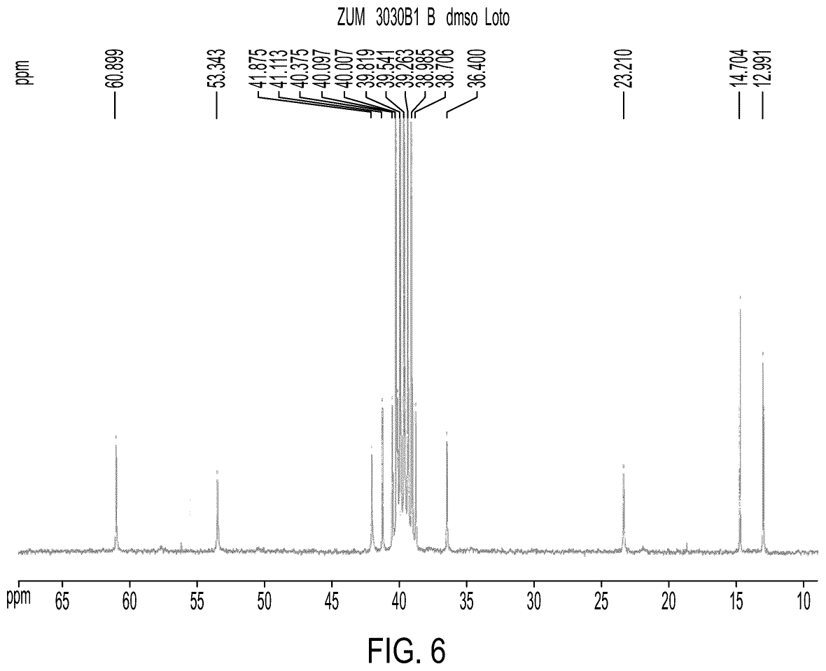

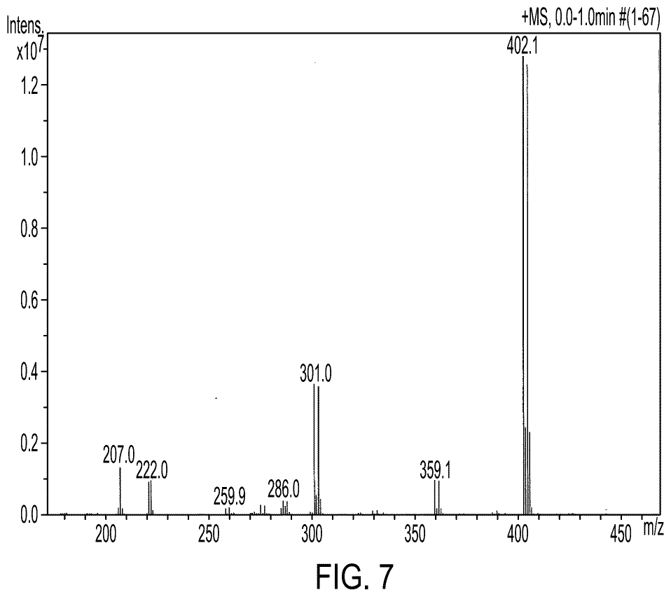

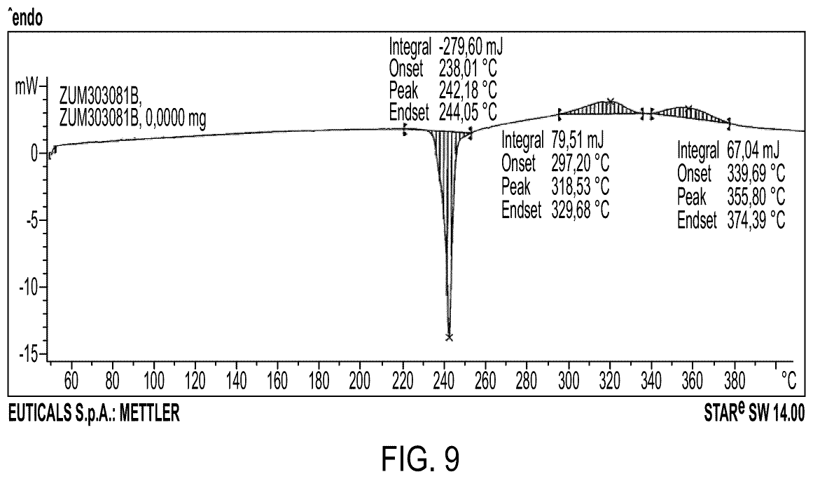

Analysis by HPLC indicated 99.45%2-bromolysergic acid diethylamide by area. The ¹H NMR spectra (FIG. 3 and FIG. 4), ¹³C NMR spectra (FIG. 5 and FIG. 6) and mass spectrum (FIG. 7, Agilent 6330 Ion Trap, [M+H]⁺ 402.17) are all in agreement with the proposed structure. The powder x-ray diffractogram in FIG. 8, for which diffraction peaks are listed in Table 5, was collected using a Bruker D2 X-ray Generator (30 kV, 10 mA, scan interval 3-35°, Step 0.04°, 2 see/step; flat plate zero-background sample holder, rotation 15 rpm). Differential scanning calorimetry (DSC): onset at 244° C. (see, FIG. 9). Moisture by Karl Fischer titration (KF): 0.11%. Hydrogen bromide by potentiometric titration with NaOH: 16.1% (calculated for C₂₀H₂₄BrN₃O·HBr: 16.7%). Residue on ignition (ROI): 0%. Total residual solvents: 0.36% w/w. Potency=(100−KF−residual solvents−ROI)×HPLC purity=98.98%.

TABLE 5
2-Br-LSD hydrobromide (Form 1) Reference
Standard Prepared According to Example 6

| Pos. [°2Th.] | Rel. Int. [%] |
|---|---|
| 8.2 ± 0.2 | 100.0 |
| 12.7 ± 0.2 | 10.9 |
| 14.4 ± 0.2 | 11.4 |
| 16.2 ± 0.2 | 21.7 |
| 18.0 ± 0.2 | 19.4 |
| 19.2 ± 0.2 | 33.8 |
| 20.7 ± 0.2 | 22.1 |
| 22.1 ± 0.2 | 33.1 |
| 22.4 ± 0.2 | 10.6 |
| 23.4 ± 0.2 | 15.3 |
| 23.8 ± 0.2 | 15.9 |
| 24.1 ± 0.2 | 34.7 |
| 24.6 ± 0.2 | 20.6 |
| 25.4 ± 0.2 | 14.7 |
| 26.2 ± 0.2 | 19.3 |
| 27.0 ± 0.2 | 28.2 |
| 27.8 ± 0.2 | 19.1 |
| 28.1 ± 0.2 | 10.2 |
| 28.7 ± 0.2 | 13.7 |
| 30.5 ± 0.2 | 12.6 |

All patent publications and non-patent publications are indicative of the level of skill of those skilled in the art to which this disclosure pertains. All these publications are herein incorporated by reference to the same extent as if each individual publication were specifically and individually indicated as being incorporated by reference herein. Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A method of synthesizing pharmaceutical grade 2-bromolysergic acid diethylamide (2-Br-LSD), or a pharmaceutically acceptable salt thereof, comprising:
a. filtering a mixture comprising 2-Br-LSD and a solvent;
b. allowing the 2-Br-LSD to precipitate from the filtered mixture;
c. collecting the 2-Br-LSD by filtration;
d. analyzing the collected 2-Br-LSD of (c) for the presence of iso-2-Br-LSD; and
e. (1) if a batch of the collected 2-Br-LSD of (c) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or
e. (2) if a batch of the collected 2-Br-LSD of (c) fails to meet the pre-set specification for iso-2-Br-LSD, then purifying the collected 2-Br-LSD and repeating (d) and (e), or discarding the collected 2-Br-LSD.

2. The method of claim 1, wherein the pre-set specification for iso-2-Br-LSD is not more than 2%.

3. The method of claim 1, wherein the pre-set specification for iso-2-Br-LSD is not more than 0.4%.

4. The method of claim 3, further comprising at least one of the following pre-set specifications: not more than 0.05% of lysergic acid diethylamide (LSD), not more than 0.5% of iso-LSD, not more than 0.15% each of di-bromo-LSD species comprising 2,12-dibromo-LSD, 2,13-dibromo-LSD, and 2,14-dibromo-LSD, not more than 0.15% each of tri-bromo-LSD species comprising 2,12,13-tribromo-LSD, 2,12,14-tribromo-LSD, and 2,13,14-tribromo-LSD, and not more than 1% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR,9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

5. The method of claim 3, further comprising at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, not more than 0.15% each of tri-bromo-lysergic acid species comprising 2,12,13-tribromo-LSD, 2,12,14-tribromo-LSD, and 2,13,14-tribromo-LSD, and not more than 0.15% each of 2-Br-LSD dimer species comprising (6aR,6′aR,9R,9′R)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, (6aR,6′aR,9R,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide, and (6aR,6′aR,9S,9'S)-5,5′-dibromo-N9,N9,N9′,N9′-tetraethyl-7,7′-dimethyl-4,4′,6,6a,6′,6′a,7,7′,8,8′,9,9′-dodecahydro-9,9′-biindolo[4,3-fg]quinoline-9,9′-dicarboxamide.

6. The method of claim 1, wherein (a)-(e2) are performed in the absence of chromatographic purification.

7. The method of claim 1, wherein the solvent of (a) comprises an organic solvent.

8. The method of claim 7, wherein the organic solvent comprises isopropyl acetate or ethyl acetate.

9. The method of claim 7, wherein the solvent of (a) further comprises water in an amount of less than about 3.5%.

10. The method of claim 7, wherein water is added to the filtered mixture of (a) in an amount of less than about 3.5%.

11. The method of claim 10, wherein (a) is performed at or below about 20° C. to about 25° C., the filtered mixture from (a) is heated to ensure complete dissolution prior to the addition of water, and (c) is performed at or below about 20° C. to about 25° C.

12. The method of claim 1, wherein (a) is performed at a temperature above about 20° C. to about 25° C. and (c) is performed at or below about 20° C. to about 25° C.

13. The method of claim 12, wherein (a) is performed at about 30-50° C.

14. The method of claim 1, wherein the 2-Br-LSD of (a) is the reaction product of 2-bromolysergic acid, diethylamine and an amide coupling reagent.

15. The method of claim 14, wherein the 2-bromolysergic acid is converted into pharmaceutical grade 2-Br-LSD, or a pharmaceutically acceptable salt thereof, in the absence of chromatographic purification.

16. The method of claim 14, wherein the reaction is conducted in the presence of an inert solvent.

17. The method of claim 16, wherein the inert solvent is tetrahydrofuran (THF).

18. The method of claim 14, wherein the amide coupling reagent is propylphosphonic anhydride (T3P), 2-(1H-benzotriazol-1-yl)-1,1,3,3-tetramethyluronium hexafluorophosphate (Hexafluorophosphate Benzotriazole Tetramethyl Uronium, or HBTU), 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium, or HATU), 1,1′-Carbonyldiimidazole (CDI), or acetic anhydride.

19. The method of claim 14, wherein the amide coupling reagent is T3P.

20. The method of claim 14, wherein the amide coupling reagent is HBTU.

21. The method of claim 1, wherein the mixture of (a) is prepared by (a1) hydrolyzing bromocriptine to form 2-bromolysergic acid, wherein (a1) is conducted in an environment in which radical survival is inhibited, followed by (a2) contacting 2-bromolysergic acid with diethylamine and an amide coupling reagent, to form 2-Br-LSD, followed by (a3) adding the solvent used to prepare the mixture of (a).

22. The method of claim 21, wherein (a1) and (a2) are conducted in the absence of chromatographic purification.

23. The method of claim 21, wherein (a1) is conducted substantially in the absence of oxygen.

24. The method of claim 21, wherein (a1) is conducted in the presence of a radical scavenger.

25. The method of claim 21, wherein (a1) comprises contacting bromocriptine with a base in the presence of water and an organic solvent.

26. The method of claim 25, wherein the base is potassium hydroxide.

27. The method of claim 25, wherein the organic solvent is methanol.

28. A method of synthesizing 2-bromolysergic acid according to Good Manufacturing Practice (GMP) as an Active Pharmaceutical Ingredient (API) or as an intermediate for API manufacturing, comprising hydrolyzing bromocriptine in an environment in which radical survival is inhibited.

29. The method of claim 28, wherein the hydrolyzing is conducted substantially in the absence of oxygen.

30. The method of claim 28, wherein the hydrolyzing is conducted in the presence of a radical scavenger.

31. The method of claim 28, wherein the hydrolyzing comprises contacting bromocriptine with a base in the presence of water and an organic solvent.

32. The method of claim 31, wherein the base is potassium hydroxide.

33. The method of claim 31, wherein the organic solvent is methanol.

34. A batch of pharmaceutical grade 2-Br-LSD, or a pharmaceutically acceptable salt thereof, prepared according to the method of claim 1.

35. A batch of 2-bromolysergic acid, or a pharmaceutically acceptable salt thereof, prepared according to the method of claim 28.

36. A batch of 2-bromolysergic acid that:
a. contains not more than 0.15% each of di-bromo-lysergic acid species comprising 2,12-dibromo-lysergic acid, 2,13-dibromo-lysergic acid, and 2,14-dibromo-lysergic acid, and tri-bromo-lysergic acid species comprising 2,12,13-tribromo-lysergic acid, 2,12,14-tribromo-lysergic acid, and 2,13,14-tribromo-lysergic acid;
b. contains not more than 0.05% of lysergic acid;
c. contains not more than 1% each of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid;
d. contains not more than 6% of iso-2-bromolysergic acid; and
e. contains not less than 90% of 2-bromolysergic acid.

37. The batch of claim 36, that contains not more than 0.1% each of 2,12-dibromo-lysergic acid, 2,13-dibromo-lysergic acid, and 2,14-dibromo-lysergic acid, and not more than 0.05% each of 2,12,13-tribromo-lysergic acid, 2,12,14-tribromo-lysergic acid, and 2,13,14-tribromo-lysergic acid.

38. The batch of claim 36, that contains not more than 0.05% each of 2,12-dibromo-lysergic acid, 2,13-dibromo-lysergic acid, 2,14-dibromo-lysergic acid, 2,12,13-tribromo-lysergic acid, 2,12,14-tribromo-lysergic acid, and 2,13,14-tribromo-lysergic acid.

39. The batch of claim 36, that contains not more than 0.5% each of (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid.

40. The batch of claim 36, wherein total amount of 2-bromolysergic acid dimer species comprising (6aR,6′aR,9R,9′R)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, (6aR,6′aR,9R,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid, and (6aR,6′aR,9S,9'S)-5,5′-dibromo-7,7′-dimethyl-4,4′,6,6a,6′,6′a,7,7′,8,8′,9,9′-dodecahydro-9,9′-biindolo[4,3-fg]quinoline-9,9′-dicarboxylic acid is not more than 0.5%.

41. The batch of claim 36, that contains not more than 4% of iso-2-bromolysergic acid.

42. The batch of claim 36, that contains not less than 94% of 2-bromolysergic acid.

43. The batch of claim 42, that contains not less than 96% of 2-bromolysergic acid.

44. The batch of claim 36 that is manufactured according to GMP as an intermediate for API manufacturing or as an API.

45. A pharmaceutical composition, comprising the batch of claim 34, or a portion thereof, and a pharmaceutically acceptable carrier.
